(19) United States
(12) Patent Application Publication
(10) Pub. No.: US 2025/0304542 A1
(43) Pub. Date: Oct. 2, 2025

(54) Fused Heterobicyclic Antiviral Agents

(71) Applicant: Enanta Pharmaceuticals, Inc., Watertown, MA (US)

(72) Inventors: Sean RAFFERTY, Watertown, MA (US); William CASSELS, Watertown, MA (US); Samuel BARTLETT, Brighton, MA (US); Nathaniel Thomas KENTON, Watertown, MA (US); Jonathan THIELMAN, Cambridge, MA (US); Joseph D. PANARESE, Newton, MA (US); Scott MITCHELL, Woburn, MA (US)

(21) Appl. No.: 19/075,410

(22) Filed: Mar. 10, 2025

Related U.S. Application Data
(60) Provisional application No. 63/563,649, filed on Mar. 11, 2024.

(51) Int. Cl. C07D 285/36; A61K 31/554; A61K 45/06; A61P 31/20; C07D 417/06; C07D 417/12

(52) U.S. Cl. CPC C07D 285/36; A61K 31/554; A61K 45/06; A61P 31/20; C07D 417/06; C07D 417/12

(57) ABSTRACT

The present invention discloses compounds of Formula (I), or pharmaceutically acceptable salts, thereof:

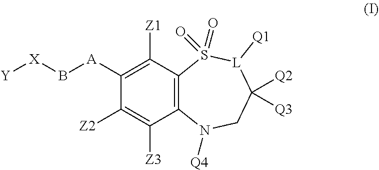

which inhibit the cellular entry of hepatitis B virus (HBV) and/or hepatitis D virus (HDV) or interfere with the function of the life cycle of HBV and/or HDV and are also useful as antiviral agents. The present invention further relates to pharmaceutical compositions comprising the aforementioned compounds for administration to a subject suffering from HBV and/or HDV infection. The invention also relates to methods of treating an HBV and/or HDV infection in a subject by administering a therapeutically effective amount of a compound of the present invention.

### RELATED APPLICATION

This application claims the benefit of U.S. Provisional Application No. 63/563,649, filed on Mar. 11, 2024. The entire teachings of the above application are incorporated herein by reference.

### TECHNICAL FIELD

The present invention relates generally to compounds and pharmaceutical compositions useful as hepatitis virus inhibitors. Specifically, the present invention relates to benzothiadiazepine compounds that are useful in treating viral infections such as hepatitis B virus (HBV) and/or hepatitis D virus (HDV). These compounds can function through inhibition of the Na⁺-taurocholate cotransporting polypeptide (NTCP) receptor. The invention provides novel benzothiadiazepine compounds as disclosed herein, pharmaceutical compositions containing such compounds, and methods of using these compounds and compositions in the treatment and prevention of HBV and/or HDV infections.

### BACKGROUND OF THE INVENTION

The hepatitis delta viruses, or HDV, are eight species of negative-sense single-stranded RNA viruses (or virus-like particles) classified together as the genus Deltavirus, within the realm Ribozyviria. The HDV virion is a small, spherical, enveloped particle with a 36 nm diameter; its viral envelope contains host phospholipids, as well as three proteins taken from the hepatitis B virus—the large, medium, and small hepatitis B surface antigens. This assembly surrounds an inner ribonucleoprotein (RNP) particle, which contains the genome surrounded by hepatitis D antigen (HDAg).

The HDV genome is negative sense, single-stranded, closed circular RNA; with a genome of approximately 1700 nucleotides, HDV is the smallest virus known to infect animals. Its genome is unique among animal viruses because of its high GC nucleotide content. Its nucleotide sequence is about 70% self-complementary, allowing the genome to form a partially double-stranded, rod-like RNA structure. Millions of people throughout the world are chronically infected with hepatitis D virus (HDV). For those that are chronically infected, many will develop complications of liver disease from cirrhosis or hepatocellular carcinoma (HCC).

HBV is a member of the Hepadnavirus family, and it is able to replicate through the reverse transcription of an RNA intermediate. The 3.2-kb HBV genome exists in a circular, partially doublestranded DNA conformation (rcDNA) that has four overlapping open reading frames (ORF). These encode for the core, polymerase, envelope, and X proteins of the virus. rcDNA must be converted into covalently closed circular DNA (cccDNA) in cells prior to the transcription of viral RNAs. As rcDNA is transcriptionally inert, cccDNA is the only template for HBV transcription, and its existence is required for infection.

The HBV viral envelope contains a mixture of surface antigen proteins (HBsAg). The HBsAg coat contains three proteins that share a common region that includes the smallest of the three proteins (SHBsAg). The other two proteins, Medium HBsAg (MHBsAg) and Large HBsAg (LHBsAg), both contain a segment of SHBsAg with additional polypeptide segments. SHBsAg, MHBsAg, and LHBsAg can also assemble into a non-infectious subviral particle known as the 22-nm particle that contains the same proteins found around infectious viral particles. As the 22-nm particles contain the same antigenic surface proteins that exist around the infectious HBV virion, they can be used as a vaccine to produce neutralizing antibodies.

HBV and HDV both gain entry into liver cells via the human NTCP bile acid transporter. Viral particles recognize their receptor via the N-terminal domain of the large hepatitis B surface antigen, HBsAg. After entering the hepatocyte, the virus is uncoated and the nucleocapsid translocated to the nucleus thereby infecting the cell. An NTCP inhibitor derived from the pre-S1 region of large HBsAg has been conditionally approved in the European Union for the treatment of chronic HDV infection. Although small molecule NTCP inhibitors are known, none have been approved for the treatment of HDV (refer to WO2019234077, WO2020161216, WO2020161217, WO2021110883, WO2021110884, WO2021110885, WO2021110886, WO2021110887, WO2022029101, WO2022117778, WO2022196858, WO2022225035, WO2022253997, WO2023164179, WO2023164181, WO2023164183, WO2023164186).

There is a need in the art for novel therapeutic agents that treat, ameliorate or prevent HBV and/or HDV infection. Administration of these therapeutic agents to an HBV and/or HDV infected patient, either as monotherapy or in combination with other HBV and/or HDV treatments or ancillary treatments, will lead to significantly improved prognosis, diminished progression of the disease, and enhanced seroconversion rates.

### SUMMARY OF THE INVENTION

The present invention relates to novel antiviral compounds, pharmaceutical compositions comprising such compounds, as well as methods to treat or prevent viral (particularly HBV and/or HDV) infection in a subject in need of such therapy with said compounds. Compounds of the present invention inhibit the entry of HBV and/or HDV or interfere with the life cycle of HBV and/or HDV and are also useful as antiviral agents. In addition, the present invention provides processes for the preparation of said compounds.

The present invention provides compounds represented by Formula (I),

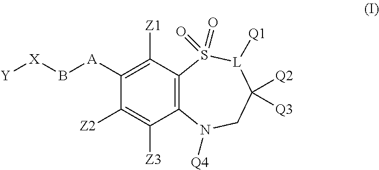

and pharmaceutically acceptable salts, N-oxides, esters and prodrugs thereof, wherein:
Q1, Q2, Q3, and Q4 are each independently selected from the group consisting of hydrogen, optionally substituted —C₁-C₆ alkyl, optionally substituted —C₂-C₆ alkenyl, optionally substituted —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, and optionally substituted heteroaryl;
Alternatively, Q1 and Q2, or Q1 and Q3 are taken together with the atoms to which they are attached to form an optionally substituted 3-8 membered heterocyclic or carbocyclic ring containing 0, 1, 2, or 3 double bonds;
Alternatively, Q2 and Q3 are taken together with the carbon atom to which they are attached to form an optionally substituted 3-8 membered heterocyclic or carbocyclic ring containing 0, 1, 2, or 3 double bonds;
L is CR₁₄ or N;
R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy;
Z1, Z2, and Z3 are each independently selected from the group consisting of:
1) hydrogen;
2) halogen;
3) —OH;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₈ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁₁)S(O)₂(R₁₂);
21) —N(R₁₁)(R₁₂);
22) —N(R₁₃)C(O)N(R₁₁)(R₁₂);
23) —N(R₁₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
wherein R₁₁, R₁₂, and R₁₃, are each independently selected from the group consisting of hydrogen, optionally substituted —C₁-C₁₂ alkyl, optionally substituted —C₂-C₁₂ alkenyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; alternatively, R₁₁ and R₁₂ are taken together with the nitrogen atom to which they are attached to form an optionally substituted 3-8 membered heterocyclic containing 0, 1, 2, or 3 double bonds;
A is absent or selected from the group consisting of O, NR₁₅, CR₁₆R₁₆′, S(O)₂, C(O), optionally substituted —C₃-C₈ cycloalkyl, optionally substituted —C₃-C₈ cycloalkenyl, and optionally substituted 3- to 8-membered heterocycloalkyl;
B is absent or selected from the group consisting of O, NR₁₅, CR₁₇R₁₇′, optionally substituted —C₃-C₈ cycloalkyl, optionally substituted —C₃-C₈ cycloalkenyl, and optionally substituted 3- to 8-membered heterocycloalkyl;
X is absent or selected from the group consisting of C(O), O, NR₁₅, SO₂, CR₁₈R₁₈′, and optionally substituted —C₃-C₈ cycloalkyl, optionally substituted —C₃-C₈ cycloalkenyl, and optionally substituted 3- to 8-membered heterocycloalkyl;
Alternatively, A is CR₁₆R₁₆′, and R₁₆ and R₁₆′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
Alternatively, B is CR₁₇R₁₇′, and R₁₇ and R₁₇′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
Alternatively, X is CR₁₈R₁₈′, and R₁₈ and R₁₈′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
Alternatively, A and B are taken together to form

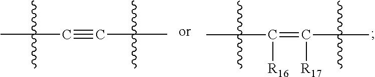

R₁₅, R₁₆, R₁₆′, R₁₇, R₁₇′, R₁₈ and R₁₈′ are each independently selected from the group consisting of:
1) hydrogen;
2) halogen;
3) hydroxy;
4) cyano;
5) —N(R₁₁)(R₁₂);
6) —N(R₁₃)C(O)N(R₁₁)(R₁₂);
7) —N(R₁₃)C(O)C(O)N(R₁₁)(R₁₂);
8) —N(R₁₁)C(O)(R₁₂);
9) —N(R₁₁)C(O)₂(R₁₂);
10) —N(R₁₃)S(O)₂N(R₁₁)(R₁₂);
11) —N(R₁₁)S(O)₂(R₁₂);
12) Optionally substituted —C₁-C₈ alkyl;
13) Optionally substituted —C₃-C₈ cycloalkyl;
14) Optionally substituted —C₃-C₈ cycloalkenyl,
15) Optionally substituted 3- to 12-membered heterocycloalkyl;
16) Optionally substituted aryl;
17) Optionally substituted heteroaryl; and
18) Optionally substituted alkoxy;
Y is —CO₂H, —PO₃H₂, —SO₃H, —B(OH)₂, —C(O)NHR₂₁, —SO₂NHR₂₁, —SO₂NHC(O)R₂₁, —C(O)NHSO₂R₂₁, tetrazolyl, or triazolyl; and
R₂₁ is independently selected from the group consisting of hydrogen, optionally substituted —C₁-C₈ alkyl, optionally substituted C₂-C₈ alkenyl, optionally substituted C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl.

### DETAILED DESCRIPTION OF THE INVENTION

In one embodiment, the present invention provides a compound of Formula (I) as described above, or a pharmaceutically acceptable salt thereof.

In certain embodiments of the compounds of Formula (I), Z1 is hydrogen, halogen, -Me, or —OMe.

In certain embodiments of the compounds of Formula (I), Z1 is hydrogen.

In certain embodiments of the compounds of Formula (I), Z3 is hydrogen, halogen, -Me, or —OMe.

In certain embodiments of the compounds of Formula (I), Z3 is hydrogen.

In certain embodiments of the compounds of Formula (I), Z1 is hydrogen, and Z3 is hydrogen.

In certain embodiments of the compounds of Formula (I), Z2 is hydrogen, halogen, —CN, —OR₁₁, or —NR₁₁R₁₂. In certain embodiments, Z2 is hydrogen, halogen, or —NHR₁₂, where R₁₂ is as previously defined and is preferably C₁-C₄-alkyl. In certain embodiments Z2 is hydrogen, chloro, fluoro, or —NH(CH₃).

In certain embodiments of the compounds of Formula (I), L is nitrogen.

In certain embodiments of the compounds of Formula (I), L is nitrogen and Q1 is hydrogen or optionally substituted methyl.

In certain embodiments of the compounds of Formula (I), L is CR₁₄, where R₁₄ is hydrogen, methyl, ethyl, isopropyl or cyclopropyl.

In certain embodiments of the compounds of Formula (I), L is CR₁₄, where R₁₄ is hydrogen, methyl, ethyl, isopropyl or cyclopropyl, and Q1 is hydrogen, or optionally substituted methyl.

In certain embodiments of the compounds of Formula (I), Q2 is hydrogen, or optionally substituted methyl.

In certain embodiments of the compounds of Formula (I), Q3 is optionally substituted —C₁-C₆ alkyl, optionally substituted —C₃-C₈ cycloalkyl, optionally substituted 3- to 8-membered heterocycloalkyl, optionally substituted aryl, or optionally substituted heteroaryl.

In certain embodiments of the compounds of Formula (I), Q3 is optionally substituted phenyl, optionally substituted benzyl, optionally substituted methyl, optionally substituted ethyl, optionally substituted n-butyl, optionally substituted t-butyl, optionally substituted isopropyl, optionally substituted isobutyl, optionally substituted neopentyl

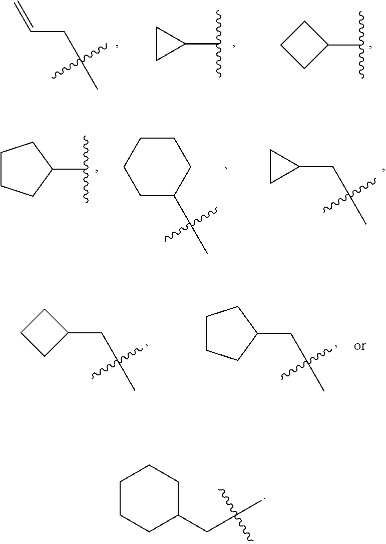

In certain embodiments of the compounds of Formula (I), Q4 is hydrogen or optionally substituted optionally substituted —C₁-C₆ alkyl.

In certain embodiments of the compounds of Formula (I), Q4 is optionally substituted —C₃-C₈ cycloalkyl or optionally substituted 3- to 8-membered heterocycloalkyl.

In certain embodiments of the compounds of Formula (I), Q4 is optionally substituted aryl or optionally substituted heteroaryl.

In certain embodiments of the compounds of Formula (I), Q4 is optionally substituted phenyl.

In certain embodiments of the compounds of Formula (I), Q4 is derived from one of the following by removal of a hydrogen atom and is optionally substituted:

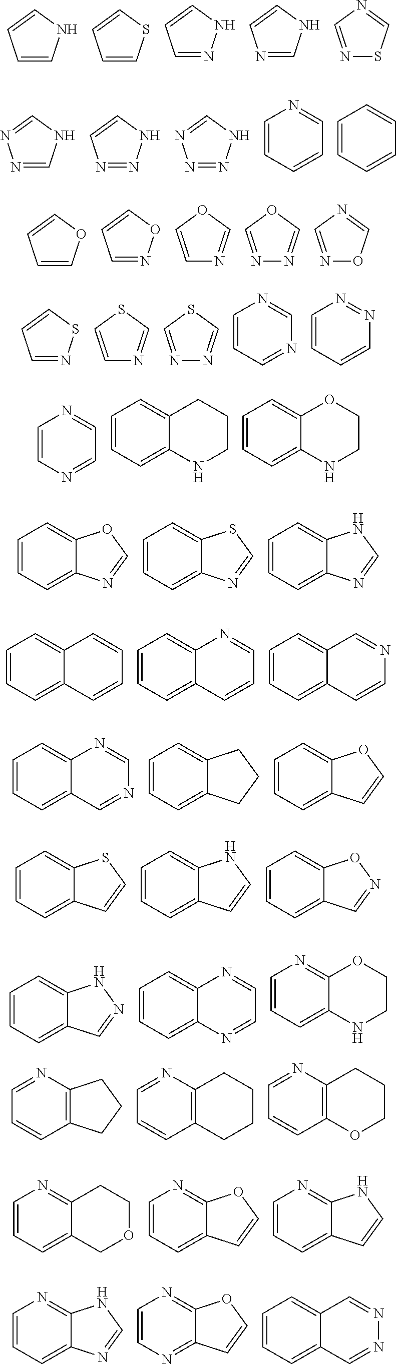

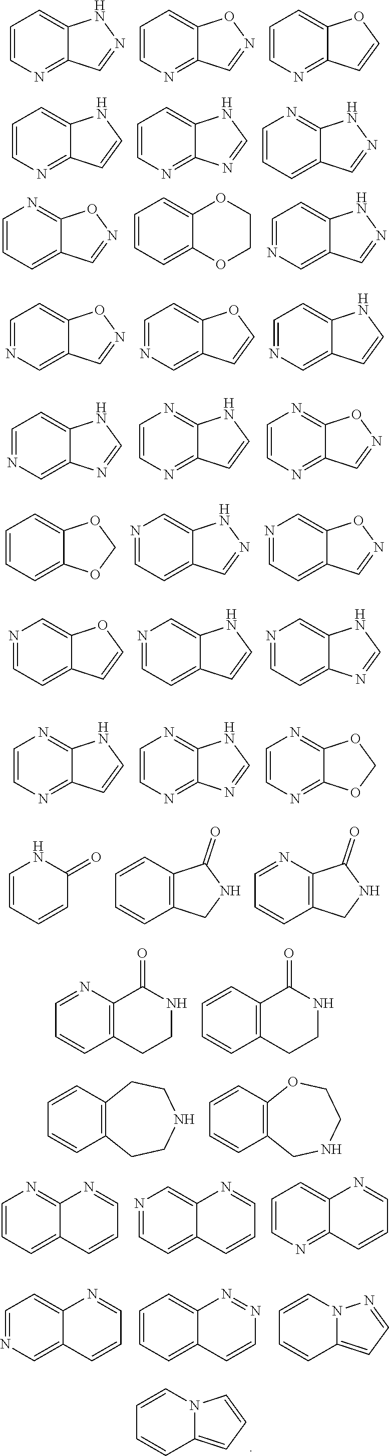

In certain embodiments of the compounds of Formula (I), Q4 is derived from one of the following by removal of a hydrogen atom and is optionally substituted:

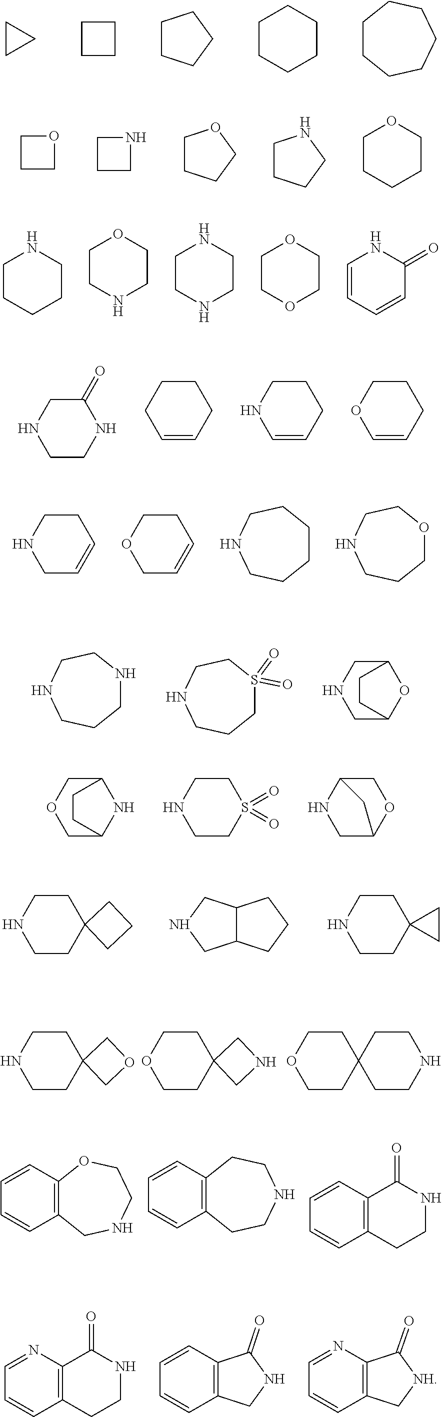

In certain embodiments of the compounds of Formula (I), A is CR₁₆R₁₆′, wherein R₁₆ and R₁₆′ are as previously defined.

In certain embodiments of the compounds of Formula (I), A is O, C(O), or CH₂.

In certain embodiments of the compounds of Formula (I), B is NR₁₅ or CR₁₇R₁₇′, wherein R₁₅, R₁₇ and R₁₇′ are as previously defined.

In certain embodiments of the compounds of Formula (I), B is NH or CH₂.

In certain embodiments of the compounds of Formula (I), A and B are taken together to form

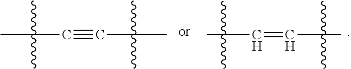

In certain embodiments of the compounds of Formula (I), A is CR₁₆R₁₆′ and B is CR₁₇R₁₇′. Preferably A and B are both CH₂.

In certain embodiments of the compounds of Formula (I), A is C(O) and B is NR₁₅, where R₁₅ is as defined above. Preferably R₁₅ is hydrogen or more, more preferably hydrogen. In these embodiments, X is preferably CR₁₈R₁₈′ and more preferably CHR₁₈, where R₁₈ and R₁₈′ are as defined above. In certain embodiments, X is CHR₁₈, where R₁₈ is optionally substituted phenyl, optionally substituted heteroaryl, optionally substituted C₃-C₈-cycloalkyl or 3 to 8-membered heterocycloalkyl.

In certain embodiments of the compounds of Formula (I), X is CR₁₈R₁₈′, wherein R₁₈ and R₁₈′ are as previously defined; preferably R₁₈ and R₁₈′ are optionally substituted —C₁-C₈ alkyl. Alternatively, R₁₈ and R₁₈′ are taken together with the carbon atoms to which they are attached to form an optionally substituted —C₃-C₈ cycloalkyl, optionally substituted —C₃-C₈ cycloalkenyl, or optionally substituted 3- to 8-membered heterocycloalkyl.

In certain embodiments of the compounds of Formula (I), X is CHR₁₈. Preferably R₁₈ is —N(R₁₁)(R₁₂), —N(R₁₁)C(O)(R₁₂), —N(R₁₁)C(O)₂(R₁₂), —N(R₁₃)C(O)N(R₁₁)(R₁₂), —N(R₁₁)S(O)₂(R₁₂), —N(R₁₃)C(O)C(O)N(R₁₁)(R₁₂), an optionally substituted —C₃-C₈ cycloalkyl, optionally substituted —C₃-C₈ cycloalkenyl, optionally substituted 3- to 8-membered heterocycloalkyl, optionally substituted aryl, or optionally substituted heteroaryl, wherein R₁₁, R₁₂ and R₁₃ are as previously defined.

In certain embodiments of the compounds of Formula (I), Y is —CO₂H.

In certain embodiments of the compounds of Formula (I), A and B are each —CH₂— or A and B are taken together to form

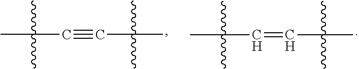

and X is CHR₁₈, where R₁₈ is as defined above. Preferably R₁₈ is —N(R₁₃)C(O)N(R₁₁)(R₁₂), —N(R₁₁)C(O)₂(R₁₂), —N(R₁₁)S(O)₂(R₁₂), or —N(R₁₃)C(O)C(O)N(R₁₁)(R₁₂), where R₁₁, R₁₂, and R₁₃ are as defined above. R₁₃ and R₁₁ are both preferably methyl or hydrogen, more preferably hydrogen. R₁₂ is preferably C₁-C₁₀-alkyl or arylalkyl, such as benzyl. Alternatively, R₁₁ and R₁₂ are taken together with the nitrogen atom to which they are attached to form a saturated 4 to 7-membered heterocycloalkyl, such as 1-pyrrolidyl or 1-piperidyl.

In certain embodiments of the compounds of Formula (I), A and B are each —CH₂— or A and B are taken together to form

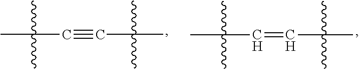

and X is CR₁₈R₁₈′. Preferably R₁₈ and R₁₈′ are independently hydrogen or C₁-C₄-alkyl. In certain embodiments, R₁₈ and R₁₈′ are independently hydrogen or methyl. In certain embodiments, R₁₈ and R₁₈′ are both hydrogen. In certain embodiments, R₁₈ and R₁₈′ are both methyl. In certain embodiments, R₁₈ and R₁₈′ are taken together with the carbon atom to which they are attached to form a C₃-C₆-cycloalkyl or a 3- to 8-membered saturated heterocycloalkyl.

In certain embodiments, the compound of Formula (I) is represented by Formula (II),

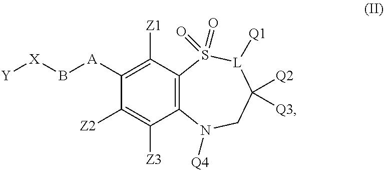

wherein Q1, Q2, Q3, Q4, Z1, Z2, Z3, A, B, X, and Y are as previously defined.

In certain embodiments, the compound of Formula (I) is represented by Formula (III),

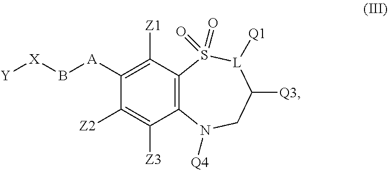

wherein L, Q1, Q3, Q4, Z1, Z2, Z3, A, B, X, and Y are as previously defined.

In certain embodiments, the compound of Formula (I) is represented by Formula (IV),

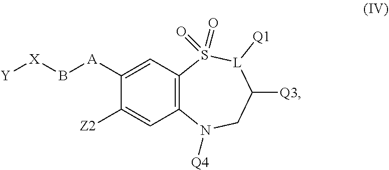

wherein L, Q1, Q3, Q4, Z2, A, B, X, and Y are as previously defined.

In certain embodiments, the compound of Formula (I) is represented by Formula (V),

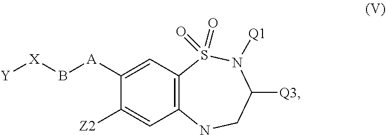

wherein Q1, Q3, Q4, Z2, A, B, X, and Y are as previously defined.

In certain embodiments, the compound of Formula (I) is represented by Formula (VI-1) or Formula (VI-2),

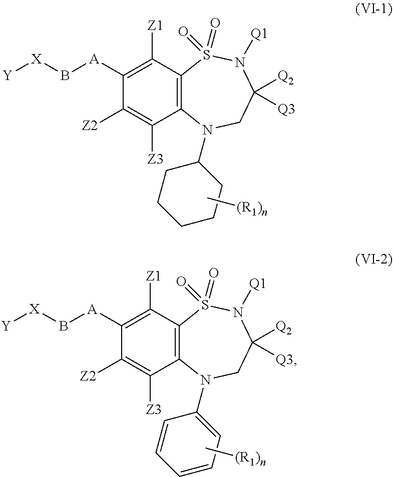

wherein each R₁ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
n is 0, 1, 2 or 3; preferably n is 0 or 1, more preferably 0; and R₁₁, R₁₂, Q1, Q2, Q3, Z1, Z2, Z3, A, B, X, and Y are as previously defined.

In certain embodiments, the compound of Formula (I) is represented by Formula (VII-1) or Formula (VII-2),

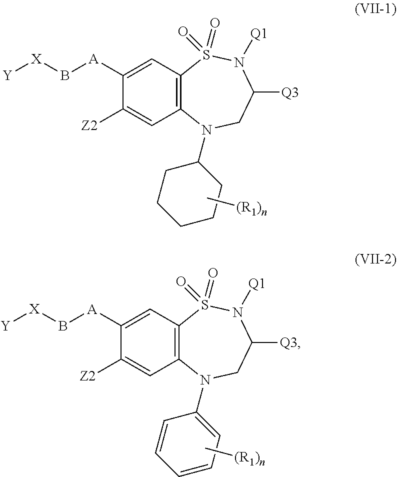

wherein n, R₁, Q1, Q3, Z2, A, B, X, and Y are as previously defined. Preferably n is 0.

In certain embodiments, the compound of Formula (I) is represented by one of Formulae (VIII-1)˜(VIII-4),

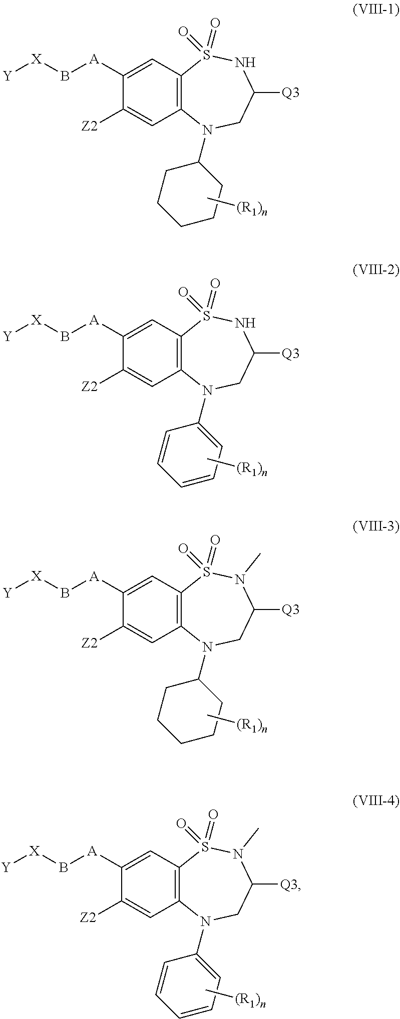

wherein n, R₁, Q3, Z2, A, B, X, and Y are as previously defined. Preferably n is 0.

In certain embodiments, the compound of Formula (I) is represented by one of Formulae (IX-1)˜(IX-4),

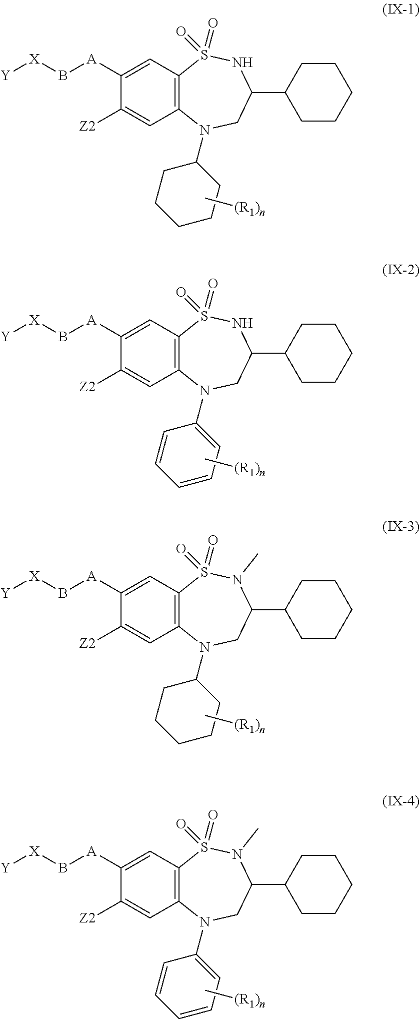

wherein n, R₁, Z2, A, B, X, and Y are as previously defined. Preferably n is 0. In certain embodiments, the compound of Formula (I) is represented by one of Formulae (X-1)˜(X-5),

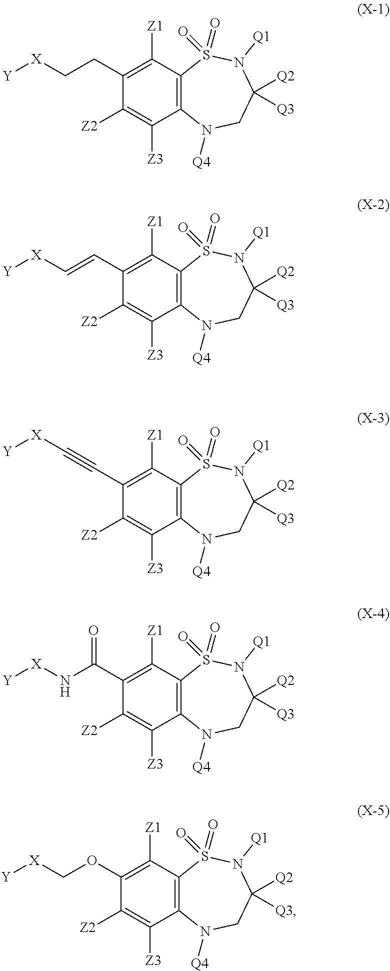

wherein Q1, Q2, Q3, Q4, Z1, Z2, Z3, X, and Y are as previously defined.

In certain embodiments, the compound of Formula (I) is represented by one of Formulae (XI-1)˜(XI-5),

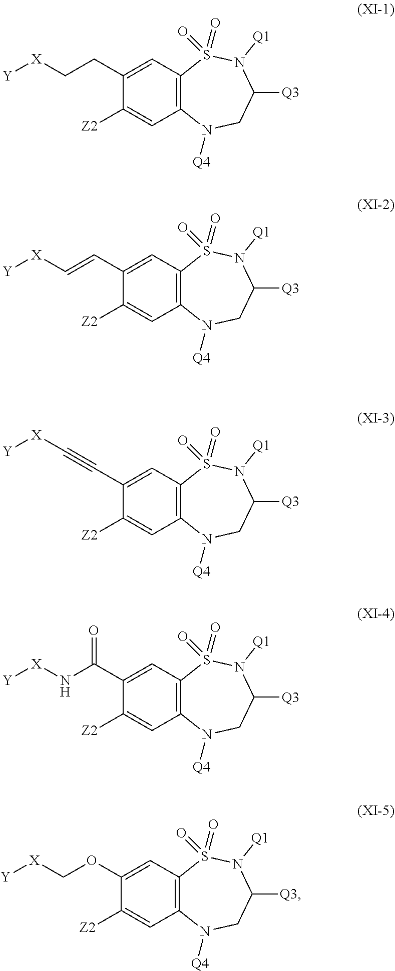

wherein Q1, Q3, Q4, Z2, X, and Y are as previously defined.

In certain embodiments, the compound of Formula (I) is represented by one of Formulae (XII-1)˜(XII-10),

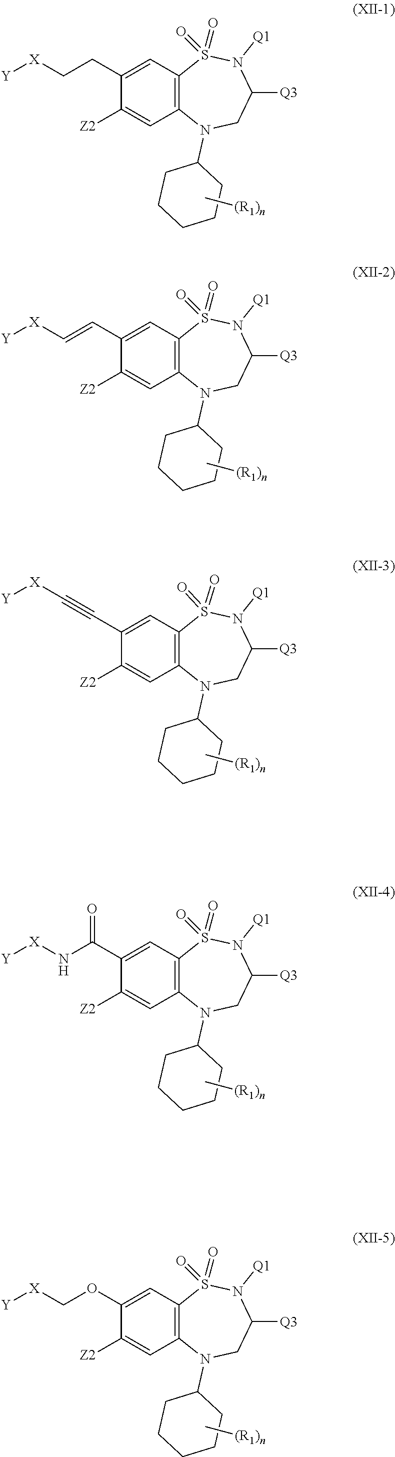

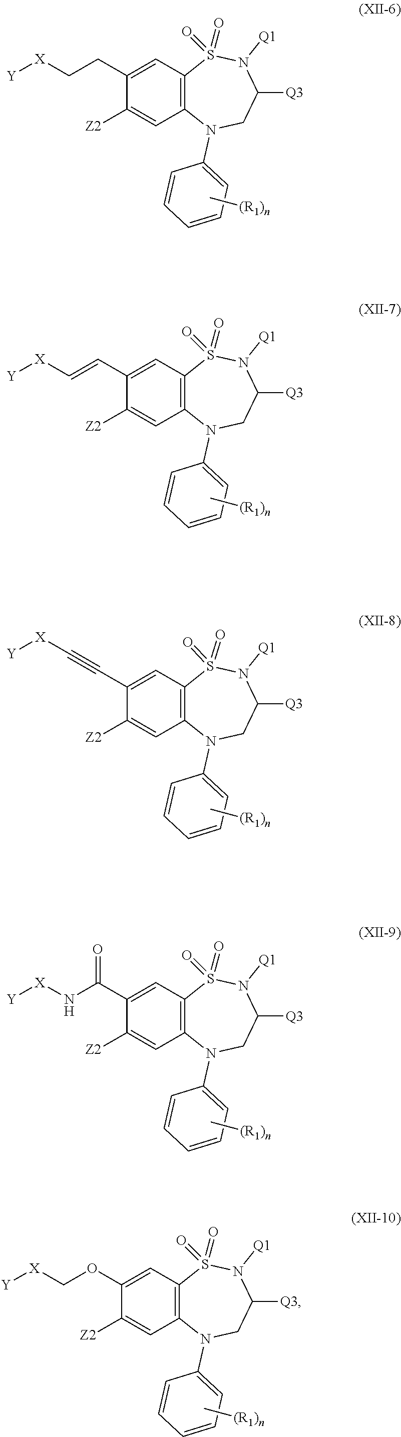

wherein n, R₁, Q1, Q3, Z2, X, and Y are as previously defined, preferably n is 0, and Z2 is hydrogen, halogen, —CN, —OR₁₁, or —NR₁₁R₁₂.

In certain embodiments, the compound of Formula (I) is represented by one of Formulae (XIII-1)˜(XIII-10),

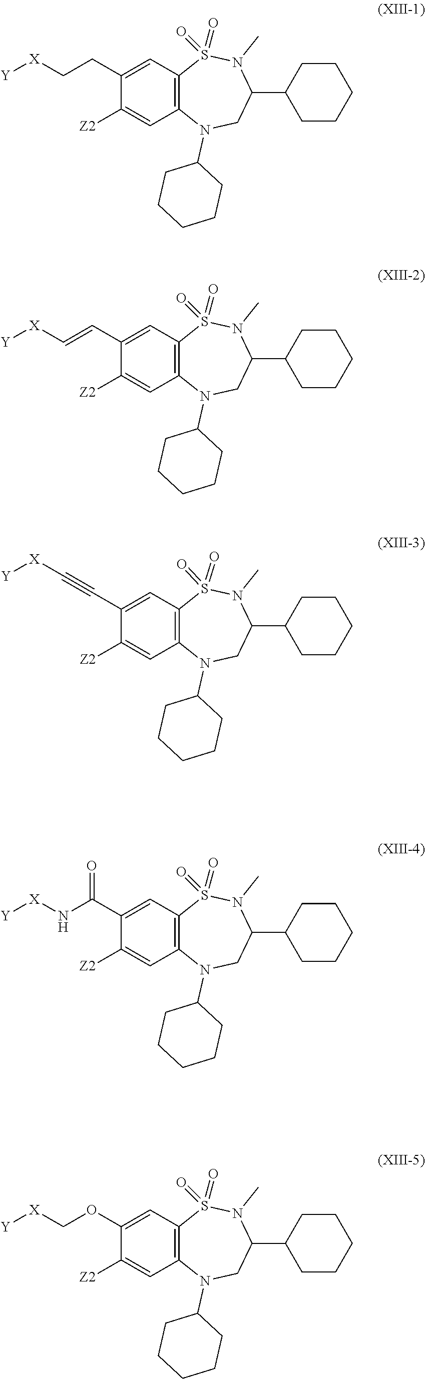

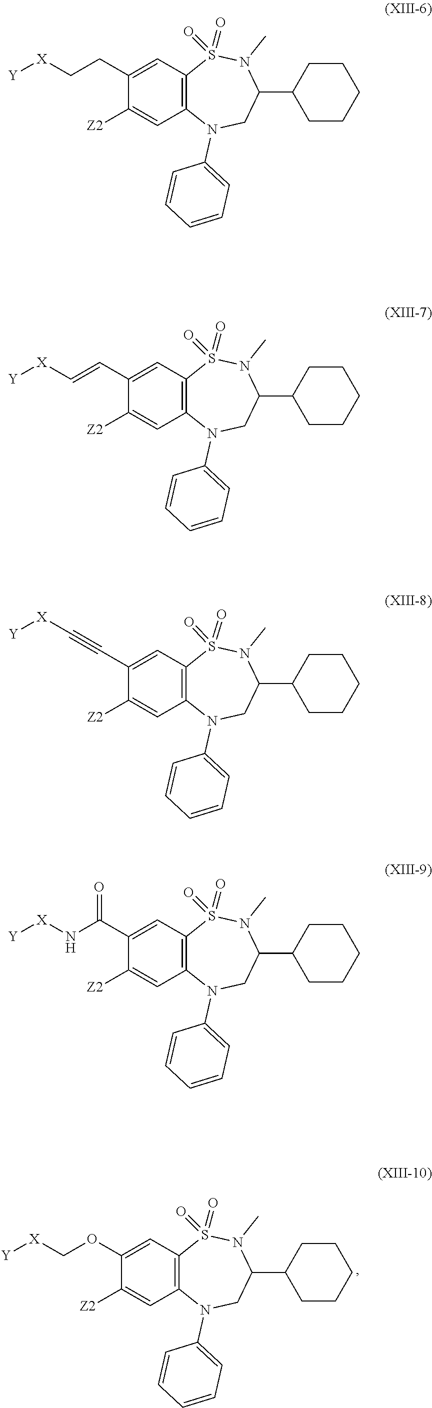

wherein Z2, X, and Y are as previously defined. Preferably Z2 is hydrogen, halogen, —CN, —OR₁₁, or —NR₁₁R₁₂.

In certain embodiments, the compound of Formula (I) is represented by one of Formulae (XIII-1)˜(XIII-10), wherein Z2 is —Cl, and Y is —COOH.

In certain embodiments, the compound of Formula (I) is represented by one of Formulae (XIII-1)˜(XIII-10), wherein Z2 is hydrogen, halogen, —CN, —OR₁₁, or —NR₁₁R₁₂, Y is —COOH, and X is CR₁₈R₁₈′. R₁₁, R₁₂, R₁₈ and R₁₈′ are previously defined. Preferably R₁₈ and R₁₈′ are optionally substituted —C₁-C₈ alkyl. Alternatively, R₁₈ and R₁₈′ are taken together with the carbon atoms to which they are attached to form an optionally substituted —C₃-C₈ cycloalkyl, optionally substituted —C₃-C₈ cycloalkenyl, or optionally substituted 3- to 8-membered heterocycloalkyl.

In certain embodiments, the compound of Formula (I) is represented by one of Formulae (XIII-1)˜(XIII-10), wherein Z2 is hydrogen, halogen, —CN, —OR₁₁, or —NR₁₁R₁₂, Y is —COOH, and X is CHR₁₈. R₁₁, R₁₂, and R₁₈ are as previously defined.

It will be appreciated that the description of the present invention herein should be construed in congruity with the laws and principles of chemical bonding. In some instances, it may be necessary to remove a hydrogen atom in order to accommodate a substituent at any given location.

It will also be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

In one embodiment, the compounds described herein are suitable for monotherapy and are effective against natural or native HBV and/or HDV strains and against HBV and/or HDV strains resistant to currently known drugs. In another embodiment, the compounds described herein are suitable for use in combination therapy.

In another embodiment, the additional therapeutic agent is selected from a core inhibitor, which includes GLS4, GLS4JHS, JNJ-379, ABI-H0731, ABI-H2158, AB-423, AB-506, WX-066, and QL-OA6A; immune modulator or immune stimulator therapies, which includes T-cell response activator AIC649 and biological agents belonging to the interferon class, such as interferon alpha 2a or 2b or modified interferons such as pegylated interferon, alpha 2a, alpha 2b, lamda; or STING (stimulator of interferon genes) modulator; or TLR modulators such as TLR-7 agonists, TLR-8 agonists or TLR-9 agonists; or therapeutic vaccines to stimulate an HBV-specific immune response such as virus-like particles composed of HBcAg and HBsAg, immune complexes of HBsAg and HBsAb, or recombinant proteins comprising HBx, HBsAg and HBcAg in the context of a yeast vector; or immunity activator such as SB-9200 of certain cellular viral RNA sensors such as RIG-I, NOD2, and MDA5 protein, or RNA interence (RNAi) or small interfering RNA (siRNA) such as ARC-520, ARC-521, ARB-1467, and ALN-HBV RNAi, or antiviral agents that block viral entry or maturation or target the HBV polymerase such as nucleoside or nucleotide or non-nucleos(t)ide polymerase inhibitors, and agents of distinct or unknown mechanism including agents that disrupt the function of other essential viral protein(s) or host proteins required for HBV replication or persistence such as REP 2139, RG7834, and AB-452. In an embodiment of the combination therapy, the reverse transcriptase inhibitor is at least one of Zidovudine, Didanosine, Zalcitabine, ddA, Stavudine, Lamivudine, Aba-cavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In another embodiment of the combination therapy, the TLR-7 agonist is selected from the group consisting of SM360320 (12-benzyl-8-hydroxy-2-(2-methoxy-ethoxy)adenine), AZD 8848 (methyl [3-({[3-(6-amino-2-butoxy-8-oxo-7,8-dihydro-9H-purin-12-yl)propyl][3-(4-morpholinyl) propyl]amino]methyl)phenyl]acetate), GS-9620 (4-Amino-2-butoxy-8-[3-(2-pyrrolidinylmethyl)benzyl]-7,8-dihydro-6 (5H)-pteridinone), AL-034 (TQ-A3334), and RO6864018.

In another embodiment of the combination therapy, the TLR-8 agonist is GS-9688.

In an embodiment of these combination therapies, the compound and the additional therapeutic agent are co-formulated. In another embodiment, the compound and the additional therapeutic agent are co-administered.

In another embodiment of the combination therapy, administering the compound of the invention allows for administering of the additional therapeutic agent at a lower dose or frequency as compared to the administering of the at least one additional therapeutic agent alone that is required to achieve similar results in prophylactically treating an HBV infection in an individual in need thereof.

In another embodiment of the combination therapy, before administering the therapeutically effective amount of the compound of the invention, the individual is known to be refractory to a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

In still another embodiment of the method, administering the compound of the invention reduces viral load in the individual to a greater extent compared to the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

In another embodiment, administering of the compound of the invention causes a lower incidence of viral mutation and/or viral resistance than the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

It should be understood that the compounds encompassed by the present invention are those that are suitably stable for use as pharmaceutical agents.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of C₁-C₈ alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of C₃-C₁₂ cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of C₃-C₁₂ cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” is a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy are C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic groups may be further substituted. Heteroaryl or Heterocyclic groups can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH— heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O— heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, CO₂—C₃-C₁₂-cycloalkyl, —CO₂-aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH— heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)— heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

It has been recognized that drug-resistant variants of HIV, HBV and HCV can emerge after prolonged treatment with an antiviral agent. Drug resistance most typically occurs by mutation of a gene that encodes for a protein such as an enzyme used in viral replication, and most typically in the case of HIV, reverse transcriptase, protease, or DNA polymerase, and in the case of HBV, DNA polymerase, or in the case of HCV, RNA polymerase, protease, or helicase. Recently, it has been demonstrated that the efficacy of a drug against HIV infection can be prolonged, augmented, or restored by administering the compound in combination or alternation with a second, and perhaps third, antiviral compound that induces a different mutation from that caused by the principal drug. The compounds can be used for combination are selected from the group consisting of a HBV polymerase inhibitor, interferon, TLR modulators such as TLR-7 agonists or TLR-9 agonists, therapeutic vaccines, immune activator of certain cellular viral RNA sensors, viral entry inhibitor, viral maturation inhibitor, distinct capsid assembly modulator, antiviral compounds of distinct or unknown mechanism, and combination thereof. Alternatively, the pharmacokinetics, biodistribution, or other parameter of the drug can be altered by such combination or alternation therapy. In general, combination therapy is typically preferred over alternation therapy because it induces multiple simultaneous stresses on the virus.

Preferred compounds for combination or alternation therapy for the treatment of HBV include 3TC, FTC, L-FMAU, interferon, adefovir dipivoxil, entecavir, telbivudine (L-dT), valtorcitabine (3′-valinyl L-dC), β-D-dioxolanyl-guanine (DXG), β-D-dioxolanyl-2,6-diaminopurine (DAPD), and β-D-dioxolanyl-6-chloropurine (ACP), famciclovir, penciclovir, lobucavir, ganciclovir, and ribavirin.

Although the invention has been described with respect to various preferred embodiments, it is not intended to be limited thereto, but rather those skilled in the art will recognize that variations and modifications may be made therein which are within the spirit of the invention and the scope of the appended claims.

### Antiviral Activity

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### Abbreviations

Abbreviations which may be used in the descriptions of the scheme and the examples that follow are: Ac for acetyl; AcOH for acetic acid; Boc₂O for di-tert-butyl-dicarbonate; Boc for t-butoxycarbonyl; Bz for benzoyl; Bn for benzyl; t-BuOK for potassium tert-butoxide; Brine for sodium chloride solution in water; CDI for carbonyldiimidazole; DCM or CH₂Cl₂ for dichloromethane; CH₃ for methyl; CH₃CN for acetonitrile; Cs₂CO₃ for cesium carbonate; CuCl for copper (I) chloride; CuI for copper (I) iodide; dba for dibenzylidene acetone; DBU for 1,8-diazabicyclo[5.4.0]-undec-7-ene; DEAD for diethylazodicarboxylate; DIAD for diisopropyl azodicarboxylate; DIPEA or (i-Pr)₂EtN for N,N,-diisopropylethyl amine; DMP or Dess-Martin periodinane for 1,1,2-tris(acetyloxy)-1,2-dihydro-1,2-benziodoxol-3-(1H)-one; DMAP for 4-dimethylamino-pyridine; DME for 1,2-dimethoxyethane; DMF for N,N-dimethylformamide; DMSO for dimethyl sulfoxide; EtOAc for ethyl acetate; EtOH for ethanol; Et₂O for diethyl ether; HATU for O-(7-azabenzotriazol-2-yl)-N,N,N′,N′,-tetramethyluronium Hexafluoro-phosphate; HCl for hydrogen chloride; K₂CO₃ for potassium carbonate; n-BuLi for n-butyl lithium; DDQ for 2,3-dichloro-5,6-dicyano-1,4-benzoquinone; LDA for lithium diisopropylamide; LiTMP for lithium 2,2,6,6-tetramethyl-piperidinate; MeOH for methanol; Mg for magnesium; MOM for methoxymethyl; Ms for mesyl or —SO₂—CH₃; NaHMDS for sodium bis(trimethylsilyl)amide; NaCl for sodium chloride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfite or sodium hydrogen sulfite; Na₂S₂O₃ for sodium thiosulfate; NH₂NH₂ for hydrazine; NH₄Cl for ammonium chloride; Ni for nickel; OH for hydroxyl; OsO₄ for osmium tetroxide; OTf for triflate; PPA for polyphophoric acid; PTSA for p-toluenesulfonic acid; PPTS for pyridinium p-toluenesulfonate; TBAF for tetrabutylammonium fluoride; TEA or Et₃N for triethylamine; TES for triethylsilyl; TESCl for triethylsilyl chloride; TESOTf for triethylsilyl trifluoromethanesulfonate; TFA for trifluoroacetic acid; THF for tetrahydrofuran; TMEDA for N,N,N′,N′-tetramethylethylene-diamine; TPP or PPh₃ for triphenyl-phosphine; Tos or Ts for tosyl or —SO₂—C₆H₄CH₃; Ts₂O for tolylsulfonic anhydride or tosyl-anhydride; TsOH for p-tolylsulfonic acid; Pd for palladium; Ph for phenyl; Pd₂(dba)₃ for tris(diben-zylideneacetone) dipalladium (0); Pd(PPh₃)₄ for tetrakis(triphenylphosphine)-palladium (0); PdCl₂(PPh₃)₂ for trans-dichlorobis-(triphenylphosphine)palladium (II); Pt for platinum; Rh for rhodium; rt for room temperature; Ru for ruthenium; TBS for tert-butyl dimethylsilyl; TMS for trimethylsilyl; or TMSCl for trimethylsilyl chloride.

### Synthetic Methods

The compounds and processes of the present invention will be better understood in connection with the following synthetic schemes that illustrate the methods by which the compounds of the invention may be prepared. These schemes are of illustrative purpose and are not meant to limit the scope of the invention. Equivalent, similar, or suitable solvents, reagents or reaction conditions may be substituted for those particular solvents, reagents, or reaction conditions described herein without departing from the general scope of the method of synthesis.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

### General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C18 column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]⁺ refers to mono-isotopic molecular weights.

NMR spectra were run on a Bruker 400 MHz spectrometer. Spectra were measured at 298K and referenced using the solvent peak. Chemical shifts for ¹H NMR are reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified. Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Ex. #1, Ex. #2, Ex. #3, Ex. #4: (R)-4-(7-chloro-3,5-dicyclohexyl-2-methyl-1,1-dioxido-2,3,4,5-tetrahydrobenzo[f][1,2,5]thiadiazepin-8-yl)-2,2-dimethylbutanoic acid

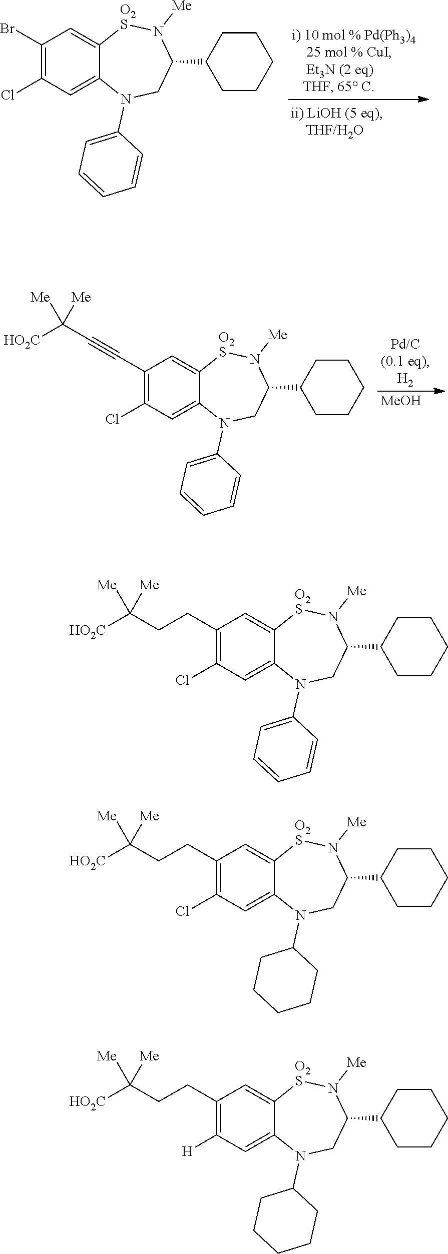

### Step 1

A solution of (R)-8-bromo-7-chloro-3-cyclohexyl-2-methyl-5-phenyl-2,3,4,5-tetrahydrobenzo[f][1,2,5]thiadiazepine 1,1-dioxide (97 mg, 0.20 mmol), copper(I) iodide (9.5 mg, 0.050 mmol) and Pd(PPh₃)₄ (23 mg, 0.020 mmol) in degassed THF (2.0 mL) was treated with methyl 2,2-dimethylbut-3-ynoate (50 mg, 0.40 mmol) and triethylamine (28 μL, 0.200 mmol). The reaction was warmed to 70° C. and stirred overnight and monitored by LCMS. Upon complete conversion, MeOH (1.0 mL) and Water (1.000 ml) were added followed by LiOH (48 mg, 2.0 mmol). The reaction was stirred until complete conversion was observed. The reaction was then quenched with 1 M aq. HCl and extracted with EtOAc. The organic layer was dried over Na₂SO₄, concentrated, and directly subjected to HPLC purification to provide (R)-4-(7-chloro-3-cyclohexyl-2-methyl-1,1-dioxido-5-phenyl-2,3,4,5-tetrahydrobenzo[f][1,2,5]thiadiazepin-8-yl)-2,2-dimethylbut-3-ynoic acid (64.5 mg, 0.125 mmol, 62.6% yield). ESI MS m/z=515.2 [M+H]⁺.

### Step 2

To a vial with (R)-4-(7-chloro-3-cyclohexyl-2-methyl-1,1-dioxido-5-phenyl-2,3,4,5-tetrahydrobenzo[f][1,2,5]thiadiazepin-8-yl)-2,2-dimethylbut-3-ynoic acid (15 mg, 0.029 mmol) and Pd/C (9.30 mg, 8.74 μmol) in MeOH (0.481 mL), a H₂ balloon was added and the reaction was stirred at room temperature. After 30 min, the reaction mixture was filtered and directly subjected to HPLC purification to provide:

(R)-4-(7-chloro-3-cyclohexyl-2-methyl-1,1-dioxido-5-phenyl-2,3,4,5-tetrahydrobenzo[f][1,2,5]thiadiazepin-8-yl)-2,2-dimethylbutanoic acid (4 mg, 7.71 mol, 26.5% yield). ESI MS m/z 519.3[M+H]⁺.

(R)-4-(7-chloro-3,5-dicyclohexyl-2-methyl-1,1-dioxido-2,3,4,5-tetrahydrobenzo[f][1,2,5]thiadiazepin-8-yl)-2,2-dimethylbutanoic acid (5 mg, 9.52 mol, 32.7% yield). ESI MS m/z=525.3[M+H]⁺.

(R)-4-(3,5-dicyclohexyl-2-methyl-1,1-dioxido-2,3,4,5-tetrahydrobenzo[f][1,2,5]thiadiazepin-8-yl)-2,2-dimethylbutanoic acid (4 mg, 8.15 mol, 28.0% yield). ESI MS m/z=491.3 [M+H]⁺.

| Ex. # | Structure | MS | NMR |
|---|---|---|---|
| 1 | 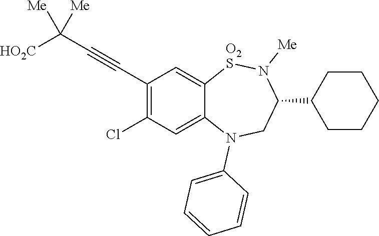 [SMILES: CN1[C@H](C2CCCCC2)CN(c2ccccc2)c2cc(Cl)c(C#CC(C)(C)C(=O)O)cc2S1(=O)=O] | [M + H]⁺ 515.2 | |
| 2 | 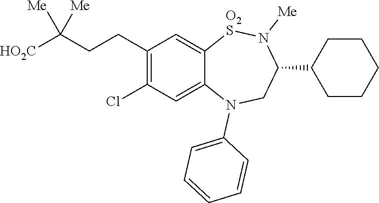 [SMILES: CN1[C@H](C2CCCCC2)CN(c2ccccc2)c2cc(Cl)c(CCC(C)(C)C(=O)O)cc2S1(=O)=O] | [M + H]⁺ 519.3 | ¹H NMR (400 MHz, CD₃CN) δ 7.80 (s, 1H), 7.31-7.22 (m, 3H), 6.90 (t, J = 7.3 Hz, 1H), 6.81 (d, J = 8.1 Hz, 2H), 4.31 (d, J = 14.8 Hz, 1H), 3.47 (d, J = 8.2 Hz, 2H), 2.83-2.74 (m, 2H), 2.61 (s, 3H), 1.89-1.75 (m, 4H), 1.72-1.64 (m, 2H), 1.63-1.54 (m, 1H), 1.36-1.29 (m, 2H), 1.27 (s, 6H), 1.24-1.11 (m, 2H), 1.09-0.95 (m, 2H). |
| 3 | 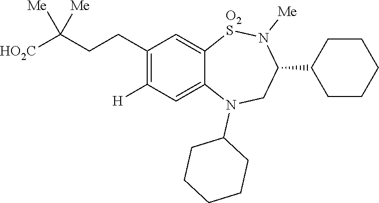 [SMILES: CN1[C@H](C2CCCCC2)CN(C2CCCCC2)c2ccc(CCC(C)(C)C(=O)O)cc2S1(=O)=O] | [M + H]⁺ 491.3 | ¹H NMR (400 MHz, CD₃CN) δ 7.50 (d, J = 2.3 Hz, 1H), 7.20 (dd, J = 8.6, 2.3 Hz, 1H), 6.96 (d, J = 8.7 Hz, 1H), 3.68-3.59 (m, 1H), 3.52-3.47 (m, 1H), 3.11-3.06 (m, 1H), 2.81 (s, 3H), 2.56-2.47 (m, 2H), 2.15-2.05 (m, 1H), 2.02-1.96 (m, 1H), 1.93-1.63 (m, 10H), 1.59- 1.32 (m, 6H), 1.23 (m, 10H), 1.11-0.97 (m, 2H). |
| 4 | 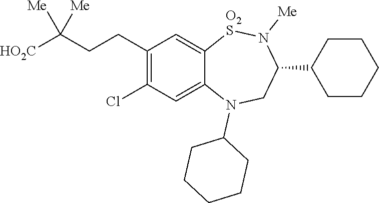 [SMILES: CN1[C@H](C2CCCCC2)CN(C2CCCCC2)c2cc(Cl)c(CCC(C)(C)C(=O)O)cc2S1(=O)=O] | [M + H]⁺ 525.3 | ¹H NMR (400 MHz, CD₃CN) δ 7.57 (s, 1H), 6.96 (s, 1H), 3.67-3.61 (m, 1H), 3.52-3.48 (m, 1H), 3.08 (m, 1H), 2.83 (s, 3H), 2.69-2.55 (m, 2H), 2.14-2.05 (m, 1H), 2.02- 1.94 (m, 2H), 1.90-1.82 (m, 8H), 1.81-1.65 (m, 5H), 1.59-1.35 (m, 4H), 1.32-1.17 (m, 5H), 1.11- 0.97 (m, 2H). |
| 5 | 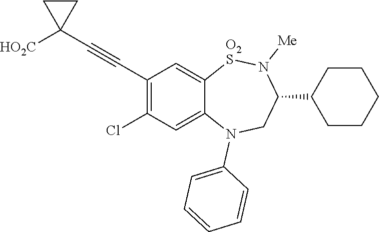 [SMILES: CN1[C@H](C2CCCCC2)CN(c2ccccc2)c2cc(Cl)c(C#CC3(C(=O)O)CC3)cc2S1(=O)=O] | [M + H]⁺ 513.3 | ¹H NMR (400 MHz, DMSO) δ 12.86 (s, 1H), 7.73 (s, 1H), 7.26 (t, J = 7.7 Hz, 2H), 7.00-6.93 (m, 4H), 4.24 (d, J = 16.0 Hz, 1H), 3.72-3.68 (m, 1H), 3.19 (s, OH), 2.63 (s, 3H), 1.87-1.79 (m, 2H), 1.62 (d, J = 9.9 Hz, 1H), 1.55-1.51 (m, 3H), 1.51- 1.44 (m, 2H), 1.39-1.30 (m, 2H), 1.19-1.00 (m, 3H), 0.95-0.81 (m, 2H) |
| 6 | 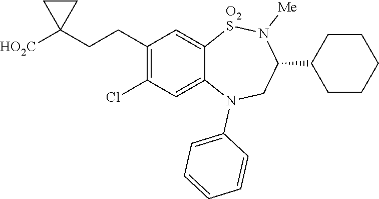 [SMILES: CN1[C@H](C2CCCCC2)CN(c2ccccc2)c2cc(Cl)c(CCC3(C(=O)O)CC3)cc2S1(=O)=O] | [M + H]⁺ 517.1 | |
| 7 | 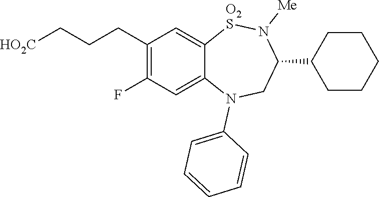 [SMILES: CN1[C@H](C2CCCCC2)CN(c2ccccc2)c2cc(F)c(CCCC(=O)O)cc2S1(=O)=O] | [M + H]⁺ 475.2 | |
| 8 | 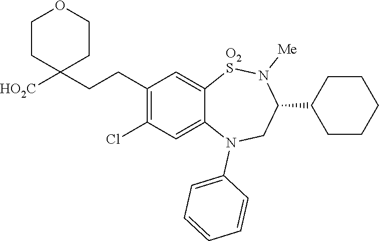 [SMILES: CN1[C@H](C2CCCCC2)CN(c2ccccc2)c2cc(Cl)c(CCC3(C(=O)O)CCOCC3)cc2S1(=O)=O] | [M + H]⁺ 567.4 | |
| 9 | 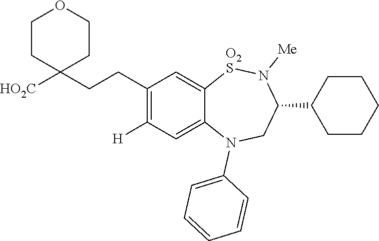 [SMILES: CN1[C@H](C2CCCCC2)CN(c2ccccc2)c2ccc(CCC3(C(=O)O)CCOCC3)cc2S1(=O)=O] | [M + H]⁺ 533.4 | |

### Ex. 10: (R,E)-4-(7-chloro-3,5-dicyclohexyl-2-methyl-1,1-dioxido-2,3,4,5-tetrahydrobenzo[f][1,2,5]thiadiazepin-8-yl)-2,2-dimethylbut-3-enoic acid

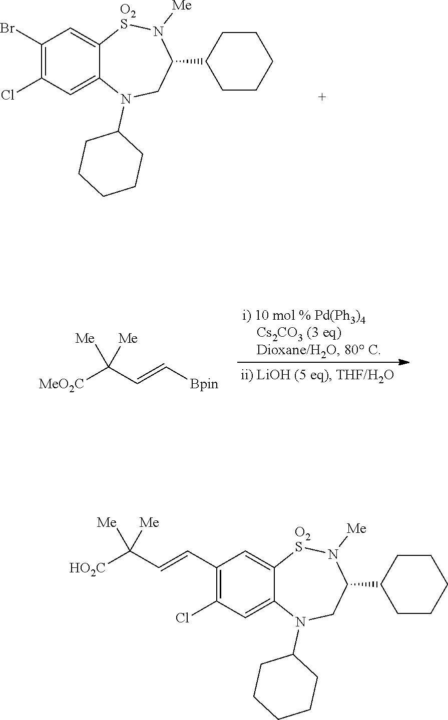

### Step 1

To a vial containing methyl (E)-2,2-dimethyl-4-(4,4,5,5-tetramethyl-1,3,2-dioxaborolan-2-yl)but-3-enoate (31 mg, 1.2 Eq, 0.12 mmol), (R)-8-bromo-7-chloro-3,5-dicyclohexyl-2-methyl-2,3,4,5-tetrahydrobenzo[f][1,2,5]thiadiazepine 1,1-dioxide (50 mg, 1 Eq, 0.10 mmol), cesium carbonate (0.10 g, 3 Eq, 0.31 mmol), and bis(triphenylphosphine)palladium(II) chloride (7.2 mg, 0.1 Eq, 10 mol) under nitrogen was added 1,4-dioxane (0.87 mL) and water (0.15 mL). The reaction was heated to 90° C. Upon completion, the reaction was cooled to room temperature, then lithium hydroxide monohydrate (21 mg, 5 Eq, 0.51 mmol) was added and the reaction was stirred until complete saponification was observed by LCMS. The mixture was then diluted with EtOAc, washed with HCl. The organic layer was concentrated and directly subjected to HPLC purification to provide (R,E)-4-(7-chloro-3,5-dicyclohexyl-2-methyl-1,1-dioxido-2,3,4,5-tetrahydrobenzo[f][1,2,5]thiadiazepin-8-yl)-2,2-dimethylbut-3-enoic acid (20.3 mg, 38.8 mol, 38%). ESI MS m/z=523.4 [M+H]⁺.

### Ex. #11: (R)-1-((3-cyclohexyl-7-((3-isopropoxybenzyl)oxy)-2-methyl-1,1-dioxido-5-phenyl-2,3,4,5-tetrahydrobenzo[f][1,2,5]thiadiazepin-8-yl)ethynyl)cyclopropane-1-carboxylic acid

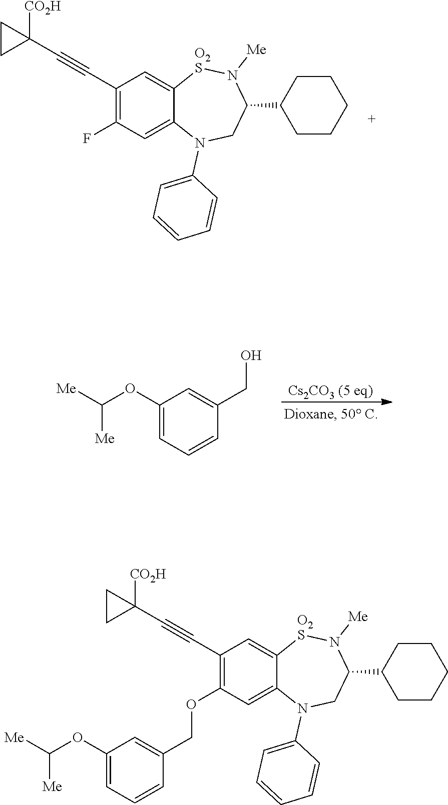 [SMILES: CN1[C@H](C2CCCCC2)CN(c2ccccc2)c2cc(F)c(C#CC3(C(=O)O)CC3)cc2S1(=O)=O ; CC(C)Oc1cccc(CO)c1 ; "Cs2CO3 (5 eq)" ; CC(C)Oc1cccc(COc2cc3c(cc2C#CC2(C(=O)O)CC2)S(=O)(=O)N(C)[C@H](C2CCCCC2)CN3c2ccccc2)c1]

### Step 1

To methyl (R)-1-((3-cyclohexyl-7-fluoro-2-methyl-1,1-dioxido-5-phenyl-2,3,4,5-tetrahydrobenzo[f][1,2,5]thiadiazepin-8-yl)ethynyl)cyclopropane-1-carboxylate (20 mg, 0.039 mmol) and Cs₂CO₃ (63.8 mg, 0.196 mmol) in Dioxane (0.402 mL), was added (3-isopropoxyphenyl)methanol (19.53 mg, 0.118 mmol). The reaction was stirred at 50° C. until complete conversion was observed by LCMS. Water (0.134 mL) was then added, followed by the addition of LiOH (9.38 mg, 0.392 mmol). The reaction mixture was stirred at room temperature until complete saponification was observed by LCMS. The reaction was quenched by the addition of formic acid (0.1 mL) and then directly subjected to HPLC purification to provide (R)-1-((3-cyclohexyl-7-((3-isopropoxybenzyl)oxy)-2-methyl-1,1-dioxido-5-phenyl-2,3,4,5-tetrahydrobenzo[f][1,2,5]thiadiazepin-8-yl)ethynyl)cyclopropane-1-carboxylic acid (12 mg, 0.019 mmol, 47.7% yield). ESI MS m/z=643.3 [M+H]⁺.

| Ex. # | Structure | MS | NMR |
|---|---|---|---|
| 11 | 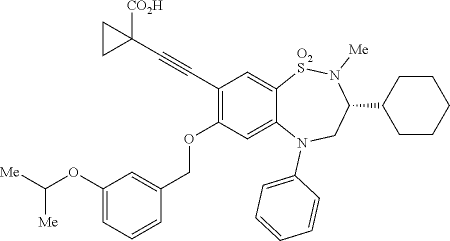 [SMILES: CC(C)Oc1cccc(COc2cc3c(cc2C#CC2(C(=O)O)CC2)S(=O)(=O)N(C)[C@H](C2CCCCC2)CN3c2ccccc2)c1] | [M + H]⁺ 643.3 | |

### Ex. 12: Synthesis of (S)-2-((tert-butoxycarbonyl)amino)-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid

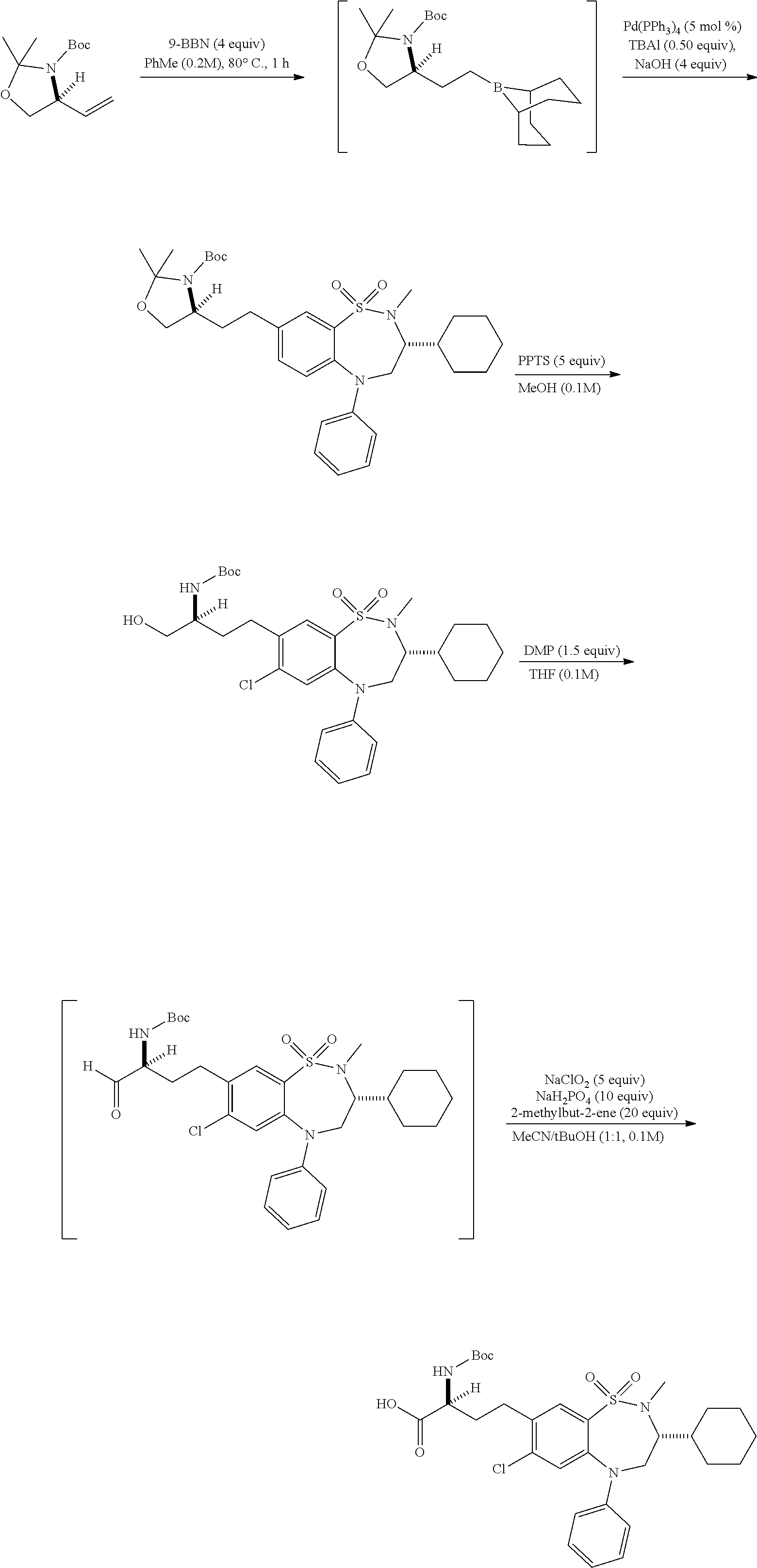

### Step 1

In a 40 mL vial equipped with a stir bar, (S)-vinyl glycinol (300 mg, 1.32 mmol, 1.0 equiv) was added under a nitrogen atmosphere and the vial was sealed with electrical tape. Next, toluene (6.0 mL, 0.2 M) was added, followed by 9-BBN (0.50 M solution in THF, 10.6 mL, 5.28 mmol, 4.0 equiv), and the reaction was heated to 80° C. for 1 h. Upon cooling to room temperature, LCMS analysis indicated full conversion to the desired hydroboration product. At room temperature, a solution of NaOH (211 mg, 5.28 mmol, 4.0 equiv) in water (1.5 mL) was added via syringe in a single portion, taking care to not introduce oxygen. Next, a solution of Pd(PPh₃)₄ (76.3 mg, 0.066 mmol, 0.05 equiv), (R)-8-bromo-7-chloro-3-cyclohexyl-2-methyl-5-phenyl-2,3,4,5-tetrahydrobenzo[f][1,2,5]thiadiazepine (702 mg, 1.45 mmol, 1.1 equiv), and tetrabutyl ammonium iodide (TBAI) (244 mg, 0.66 mmol, 0.50 equiv) in toluene (3.0 mL) was added via syringe in a single portion, taking care to not introduce oxygen. The reaction mixture was heated to 90° C. for 7 h, monitoring by LCMS. Upon complete conversion of the aryl bromide, the reaction was allowed to cool to room temperature, diluted with 5 mL each of water and EtOAc. The layers were separated and the aqueous layer was extracted 1× with EtOAc. The combined organic layers were concentrated under vacuum and purified by silica gel column chromatography (0 to 40% ethyl acetate/hexanes) to afford tert-butyl (S)-4-(2-((R)-7-chloro-3-cyclohexyl-2-methyl-1,1-dioxido-5-phenyl-2,3,4,5-tetrahydrobenzo[f][1,2,5]thiadiazepin-8-yl)ethyl)-2,2-dimethyloxazolidine-3-carboxylate (0.8310 g, 1.31 mmol, 99% yield, 94% purity by LCMS). ESI MS m/z=576.2 [M−^{t}Bu+H]⁺.

### Step 2

In a 40 mL vial equipped with a stir bar, tert-butyl (S)-4-(2-((R)-7-chloro-3-cyclohexyl-2-methyl-1,1-dioxido-5-phenyl-2,3,4,5-tetrahydrobenzo[f][1,2,5]thiadiazepin-8-yl)ethyl)-2,2-dimethyloxazolidine-3-carboxylate (0.831 g, 1.31 mmol, 1.0 equiv) was dissolved in MeOH (13.1 mL, 0.1 M). Next, pyridinium p-toluenesulfonate (PPTS) (1.65 g, 6.57 mmol, 5 equiv) was added in a single portion and the reaction was stirred for 48 h at room temperature, monitored by LCMS. Upon complete conversion of the protected amino alcohol, the reaction was diluted with 5 mL each of EtOAc and water. The layers were separated, and the aqueous layer was extracted 2× with EtOAc. The combined organic layers were concentrated and purified by silica gel column chromatography (0 to 70% ethyl acetate/hexanes) to afford tert-butyl ((S)-4-((R)-7-chloro-3-cyclohexyl-2-methyl-1,1-dioxido-5-phenyl-2,3,4,5-tetrahydrobenzo[f][1,2,5]thiadiazepin-8-yl)-1-hydroxybutan-2-yl)carbamate (0.538 g, 0.91 mmol, 69% yield). ESI MS m/z=492.0 [M−Boc+H]⁺.

### Step 3

In a 40 mL vial equipped with a stir bar, tert-butyl ((S)-4-((R)-7-chloro-3-cyclohexyl-2-methyl-1,1-dioxido-5-phenyl-2,3,4,5-tetrahydrobenzo[f][1,2,5]thiadiazepin-8-yl)-1-hydroxybutan-2-yl)carbamate (0.538 g, 0.91 mmol, 1.0 equiv) was dissolved in THF (9.0 mL, 0.1 M). Next, Dess-Martin periodinane (578 mg, 1.36 mmol, 1.5 equiv) was added in a single portion and reaction was stirred for 1 h, monitored by LCMS. Upon complete conversion of the primary alcohol, the reaction was quenched with 1 mL each of sat. aq. sodium thiosulfate and sat. aq. sodium bicarbonate. The mixture was stirred for 30 mins and extracted 2× with EtOAc. The combined organics were then concentrated and transferred to a second 40 mL vial equipped with a stir bar. To the crude aldehyde, MeCN:^{t}BuOH (1:1, 9 mL, 0.1 M), a solution of NaH₂PO₄ (1.09 g, 9.09 mmol, 10 equiv) in 3.6 mL water, and 2-methylbut-2-ene (2.0 M in THF, 18.18 mmol, 20 equiv) were added sequentially. Next, sodium hypochlorite was added in a single portion and the reaction was stirred for 1 h, monitoring by LCMS. The reaction was quenched by the addition of 5 mL water and extracted 2× with EtOAc. The combined organics were concentrated and purified by silica gel column chromatography (0 to 70% ethyl acetate/hexanes) to afford (S)-2-((tert-butoxycarbonyl)amino)-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid (0.467 g, 0.770 mmol, 85% yield). ESI MS m/z=506.0 [M-Boc+H]⁺.

The following compounds were prepared using a procedure analogous to that described for Ex. 12:

| Ex. # | Structure | MS |
|---|---|---|
| 12 | 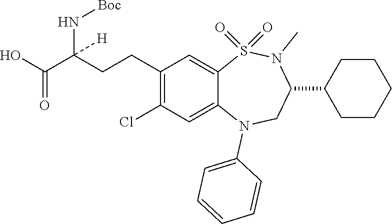 [SMILES: CN1[C@H](C2CCCCC2)CN(c2ccccc2)c2cc(Cl)c(CC[C@H](NC(=O)OC(C)(C)C)C(=O)O)cc2S1(=O)=O] | [M − Boc + H]⁺ 506.0 |
| 13 | 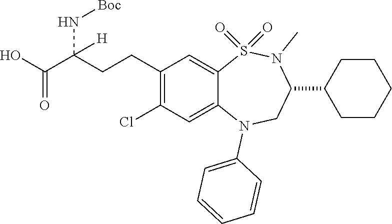 [SMILES: CN1[C@H](C2CCCCC2)CN(c2ccccc2)c2cc(Cl)c(CC[C@@H](NC(=O)OC(C)(C)C)C(=O)O)cc2S1(=O)=O] | [M − Boc + H]⁺ 506.3 |

### Ex. 14: Synthesis of (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-((isobutoxycarbonyl)amino)butanoic acid

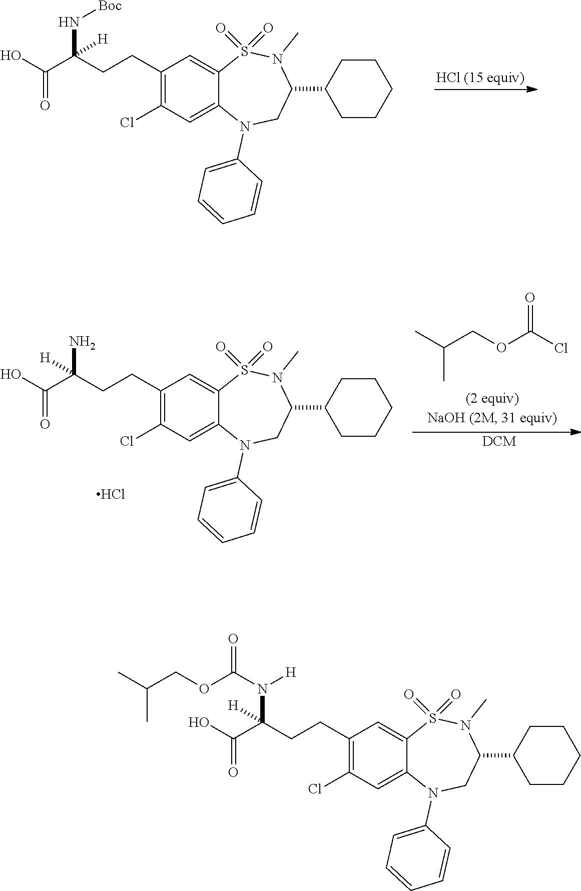

### Step 1

In a 40 mL vial equipped with a stir bar, (S)-2-((tert-butoxycarbonyl)amino)-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid (0.467 g, 0.770 mmol, 1.0 equiv) was added neat. Directly to the solid carbamate, hydrochloric acid (4.0 M in dioxanes, 2.89 mL, 11.6 mmol, 15 equiv) was added dropwise. The reaction was stirred for 30 min, monitored by LCMS. Upon complete conversion to the primary amine, the reaction was concentrated under vacuum to provide (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (0.415 g, 0.765 mmol, 99% yield). ESI MS m/z=506.4 [M+H]⁺.

### Step 2

In a 1-dram vial equipped with a stir bar, (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (7.0 mg, 0.013 mmol, 1.0 equiv) was dissolved in 1,4-dioxane (0.20 mL, 0.07 M). To the solution, sodium hydroxide (2.0 M in water, 0.20 mL, 31 equiv) was added in a single portion and the reaction was stirred. iso-Butyl chloroformate (3.3 μL, 0.026 mmol, 2 equiv) was then added in a single portion. The reaction was stirred for 2 h, monitored by LCMS. Upon complete conversion to the desired carbamate, the reaction was quenched with 0.25 mL of formic acid, diluted with 1.0 mL of DMF, filtered through a syringe filter, and purified directly via RPHPLC to provide (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-((isobutoxycarbonyl)amino)butanoic acid. ESI MS m/z=606.2 [M+H]⁺.

The following compounds were prepared using a procedure analogous to that described for Ex. 14:

| EP # | Structure | MS |
|---|---|---|
| 14 | 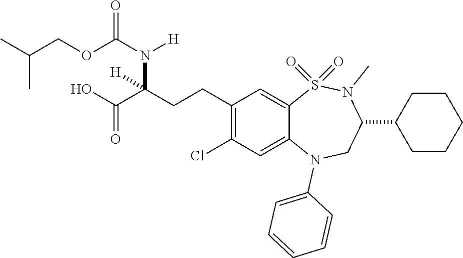 [SMILES: CC(C)COC(=O)N[C@@H](CCc1cc2c(cc1Cl)N(c1ccccc1)C[C@@H](C1CCCCC1)N(C)S2(=O)=O)C(=O)O] | [M + H]⁺ 606.2 |
| 15 | 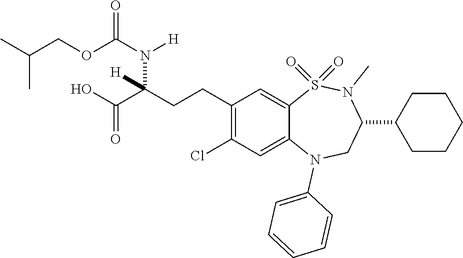 [SMILES: CC(C)COC(=O)N[C@H](CCc1cc2c(cc1Cl)N(c1ccccc1)C[C@@H](C1CCCCC1)N(C)S2(=O)=O)C(=O)O] | [M + H]⁺ 606.2 |
| 16 | 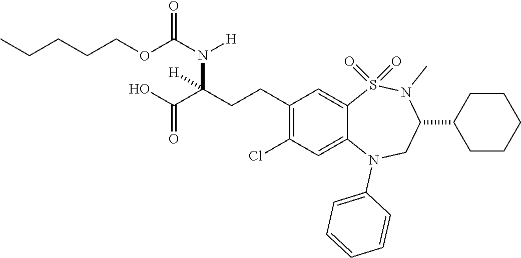 [SMILES: CCCCCOC(=O)N[C@@H](CCc1cc2c(cc1Cl)N(c1ccccc1)C[C@@H](C1CCCCC1)N(C)S2(=O)=O)C(=O)O] | [M + H]⁺ 620.0 |
| 17 | 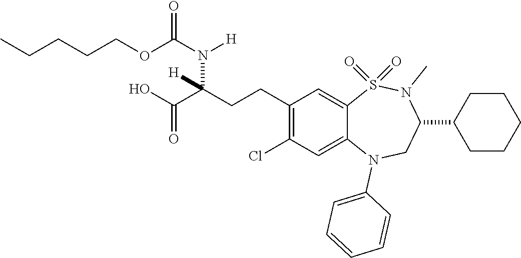 [SMILES: CCCCCOC(=O)N[C@H](CCc1cc2c(cc1Cl)N(c1ccccc1)C[C@@H](C1CCCCC1)N(C)S2(=O)=O)C(=O)O] | [M + H]⁺ 620.2 |
| 18 | 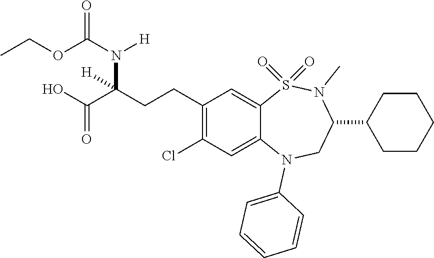 [SMILES: CCOC(=O)N[C@@H](CCc1cc2c(cc1Cl)N(c1ccccc1)C[C@@H](C1CCCCC1)N(C)S2(=O)=O)C(=O)O] | [M + H]⁺ 578.4 |
| 19 | 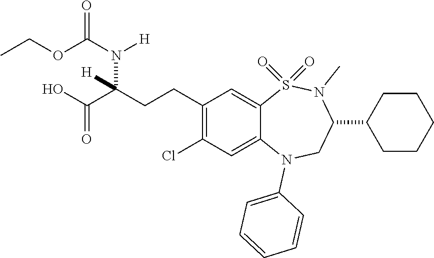 [SMILES: CCOC(=O)N[C@H](CCc1cc2c(cc1Cl)N(c1ccccc1)C[C@@H](C1CCCCC1)N(C)S2(=O)=O)C(=O)O] | [M + H]⁺ 578.3 |
| 20 | 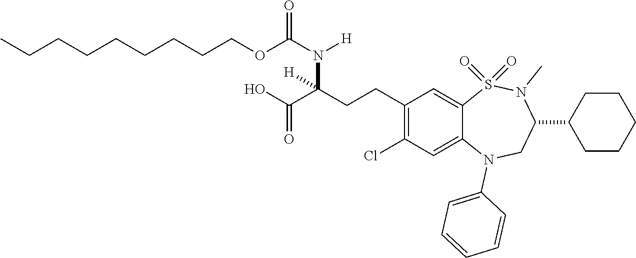 [SMILES: CCCCCCCCCOC(=O)N[C@@H](CCc1cc2c(cc1Cl)N(c1ccccc1)C[C@@H](C1CCCCC1)N(C)S2(=O)=O)C(=O)O] | [M + H]⁺ 676.4 |
| 21 | 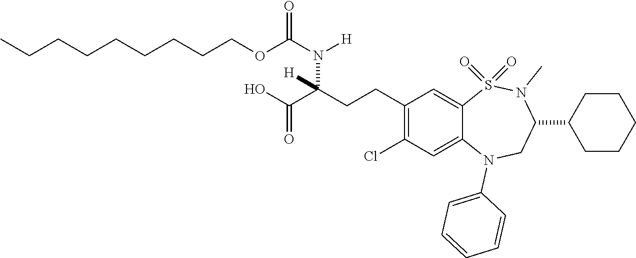 [SMILES: CCCCCCCCCOC(=O)N[C@H](CCc1cc2c(cc1Cl)N(c1ccccc1)C[C@@H](C1CCCCC1)N(C)S2(=O)=O)C(=O)O] | [M + H]+ 676.5 |
| 22 | 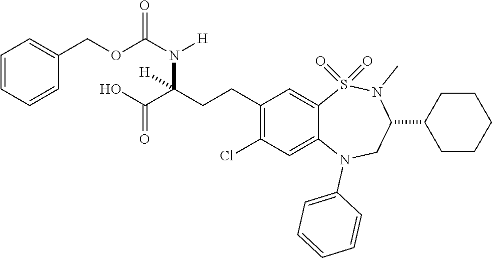 [SMILES: CN1[C@H](C2CCCCC2)CN(c2ccccc2)c2cc(Cl)c(CC[C@H](NC(=O)OCc3ccccc3)C(=O)O)cc2S1(=O)=O] | [M + H]⁺ 640.3 |
| 23 | 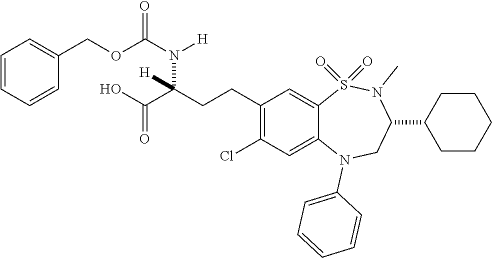 [SMILES: CN1[C@H](C2CCCCC2)CN(c2ccccc2)c2cc(Cl)c(CC[C@@H](NC(=O)OCc3ccccc3)C(=O)O)cc2S1(=O)=O] | [M + H]⁺ 640.2 |

### Ex. 24: Synthesis of (R)-4-((R)-7-chloro-3-cyclohexyl-2-methyl-1,1-dioxido-5-phenyl-2,3,4,5-tetrahydrobenzo[f][1,2,5]thiadiazepin-8-yl)-2-(piperidine-1-carboxamido)butanoic acid

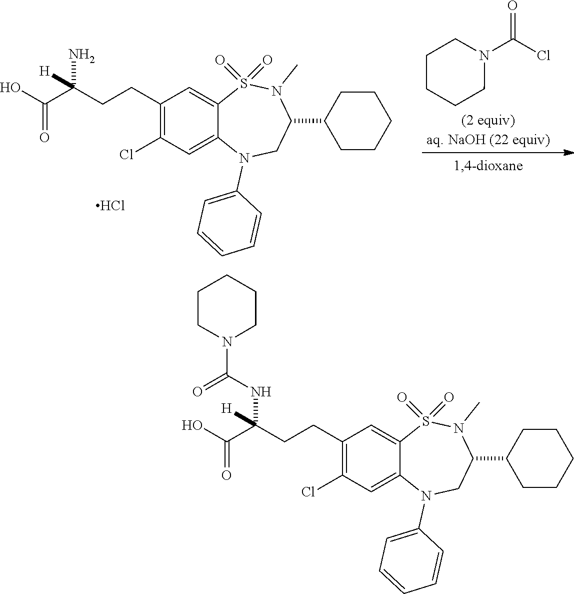

### Step 1

In a 1 dram vial equipped with a stir bar, (R)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (10.0 mg, 0.018 mmol, 1.0 equiv) was dissolved in 1,4-dioxane (0.20 mL, 0.09 M). To the solution, sodium hydroxide (2.0 M in water, 0.20 mL, 22 equiv) was added in a single portion and the reaction was stirred. Piperidine-1-carbonyl chloride (4.6 μL, 0.037 mmol, 2 equiv) was then added in a single portion. The reaction was stirred for 45 min, monitored by LCMS. A second portion of piperidine-1-carbonyl chloride (4.6 μL, 0.037 mmol, 2 equiv) was then added in a single portion and reaction was stirred for an additional 30 min. Upon sufficient conversion to the desired urea, the reaction was quenched with 0.25 mL of formic acid, diluted with 1.0 mL of DMF, filtered through a syringe filter, and purified directly via RPHPLC to provide (R)-4-((R)-7-chloro-3-cyclohexyl-2-methyl-1,1-dioxido-5-phenyl-2,3,4,5-tetrahydrobenzo[f][1,2,5]thiadiazepin-8-yl)-2-(piperidine-1-carboxamido)butanoic acid. ESI MS m/z=617.4 [M+H]⁺.

### Ex. 25: Synthesis of (R)-4-((R)-7-chloro-3-cyclohexyl-2-methyl-1,1-dioxido-5-phenyl-2,3,4,5-tetrahydrobenzo[f][1,2,5]thiadiazepin-8-yl)-2-(3-ethyl-3-methylureido)butanoic acid

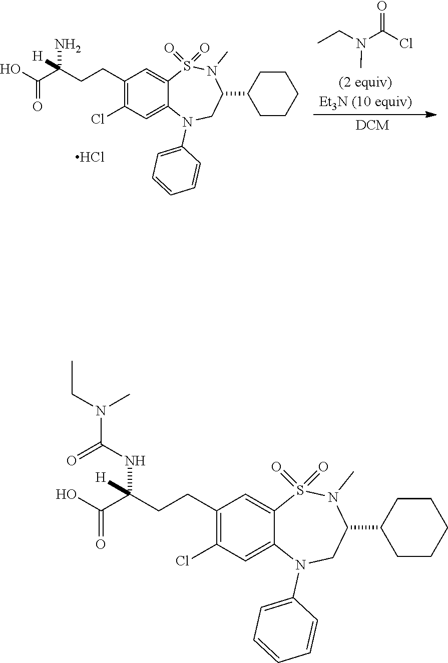

### Step 1

In a 1 dram vial equipped with a stir bar, (R)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (10.0 mg, 0.018 mmol, 1.0 equiv) was dissolved in DCM (0.4 mL, 0.05 M). To the reaction mixture, ethyl(methyl)carbamic chloride (4.5 mg, 0.036 mmol, 2.0 equiv) was added in a single portion. Then, triethylamine (0.051 mL, 0.369 mmol, 20 equiv) was added in a single portion. The reaction was stirred overnight, monitored by LCMS. Upon complete conversion to the desired urea, the reaction was quenched with 0.25 mL of formic acid, diluted with 1.0 mL of DMF, filtered through a syringe filter, and purified directly via RPHPLC to provide (R)-4-((R)-7-chloro-3-cyclohexyl-2-methyl-1,1-dioxido-5-phenyl-2,3,4,5-tetrahydrobenzo[f][1,2,5]thiadiazepin-8-yl)-2-(3-ethyl-3-methylureido)butanoic acid. ESI MS m/z=591.4 [M+H]⁺.

The following compounds were prepared using a procedure analogous to that described for Ex. 25:

| Ex. # | Structure | MS |
|---|---|---|
| 26 | 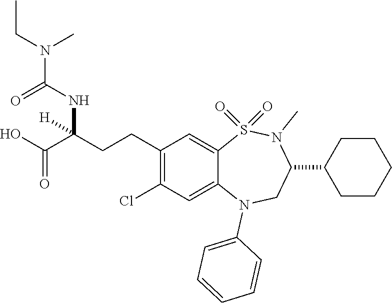 [SMILES: CCN(C)C(=O)N[C@@H](CCc1cc2c(cc1Cl)N(c1ccccc1)C[C@@H](C1CCCCC1)N(C)S2(=O)=O)C(=O)O] | [M + H]⁺ 591.4 |
| 27 | 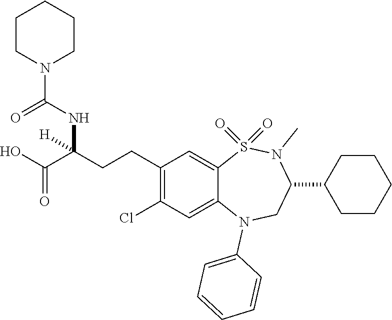 [SMILES: CN1[C@H](C2CCCCC2)CN(c2ccccc2)c2cc(Cl)c(CC[C@H](NC(=O)N3CCCCC3)C(=O)O)cc2S1(=O)=O] | [M + H]⁺ 617.0 |

### Ex. 28: Synthesis of(S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-((2-methylpropyl)sulfonamido)butanoic acid

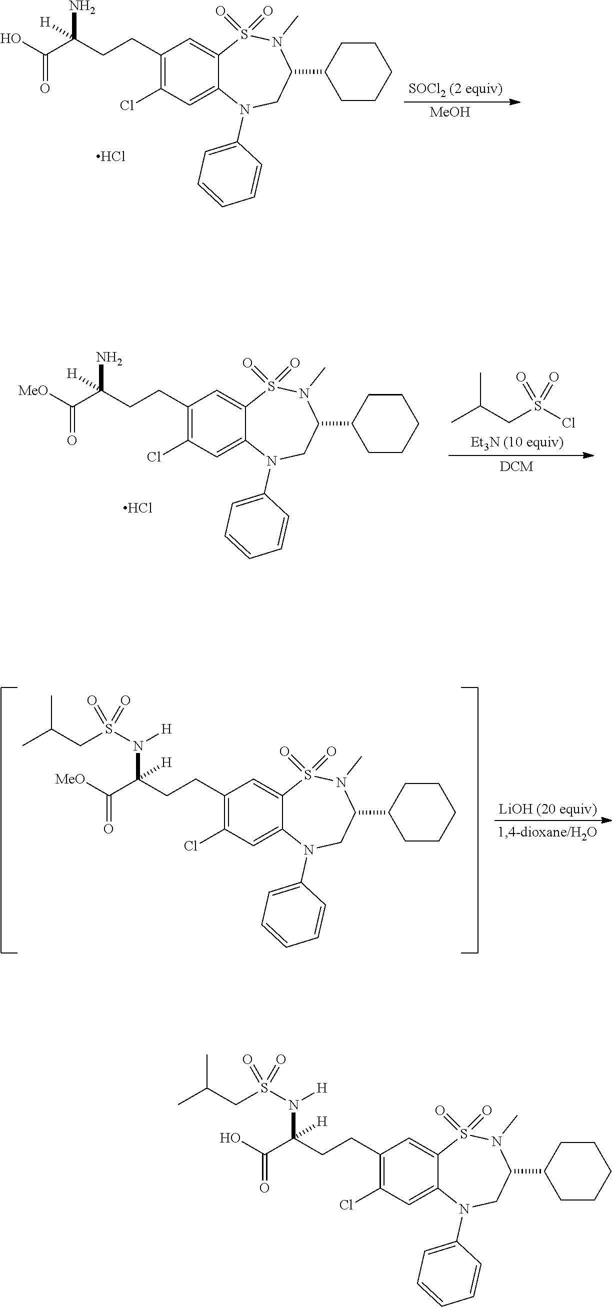

### Step 1

In a 8 mL vial equipped with a stir bar, (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (100 mg, 0.184 mmol, 1.0 equiv) was dissolved in MeOH (1.5 mL, 0.12 M). To the reaction mixture, thionyl chloride (27 μL, 0.369 mmol, 2.0 equiv) was added dropwise and the reaction was stirred for 4 h at room temperature, monitored by LCMS. Upon complete conversion to the methyl ester, the solvent was removed under vacuum. The crude material was lyophilized from MeCN/H₂O to provide methyl (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoate hydrochloride (90.0 mg, 0.162 mmol, 88% yield) as an off-white solid that was used without further purification. ESI MS m/z=520.5 [M+H]⁺.

### Step 2

In a 1-dram vial equipped with a stir bar, methyl (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoate hydrochloride (10.0 mg, 0.018 mmol, 1.0 equiv) was dissolved in DCM (0.50 mL, 0.03 M). To the reaction mixture, a solution of iso-butyl sulfonyl chloride (14.1 mg, 0.090 mmol, 5.0 equiv) in DCM (0.25 mL) was added dropwise. Then, Et₃N (25 μL, 0.18 mmol, 10 equiv) was added dropwise to the reaction mixture. The reaction was stirred for 30 min, monitored by LCMS. Upon complete conversion to the sulfonamide, the solvent was removed under a stream of N₂ to provide crude methyl (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-((2-methylpropyl)sulfonamido)butanoate (ESI MS m/z=640.5 [M+H]⁺). To the crude material, 1,4-dioxane (0.50 mL, 0.03 M), water (0.25 mL), and lithium hydroxide (8.6 mg, 0.36 mmol, 20 equiv) were added in that order. The reaction mixture was aggressively stirred at room temperature for 2 h, monitored by LCMS. Upon complete conversion to the acid, the reaction was quenched with 0.25 mL of formic acid, diluted with 1.0 mL of DMF, filtered through a syringe filter, and purified directly via RPHPLC to provide (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-((2-methylpropyl)sulfonamido)butanoic acid. ESI MS m/z=626.4 [M+H]⁺.

The following compounds were prepared using a procedure analogous to that described for Ex. 28:

| Ex. # | Structure | MS |
|---|---|---|
| 29 | 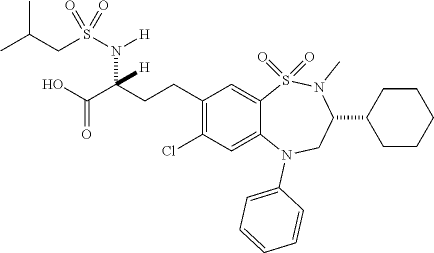 [SMILES: CC(C)CS(=O)(=O)N[C@H](CCc1cc2c(cc1Cl)N(c1ccccc1)C[C@@H](C1CCCCC1)N(C)S2(=O)=O)C(=O)O] | [M + H]⁺ 626.3 |
| 30 | 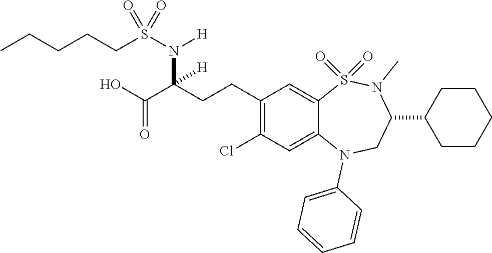 | [M + H]⁺ 654.2 |
| 31 | 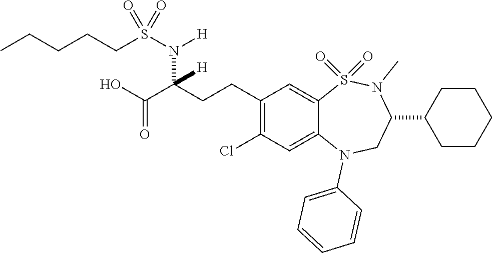 | [M + H]⁺ 654.4 |

### Ex. 32: Synthesis of (S)-2-(2-(butyl(methyl)amino)-2-oxoacetamido)-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid

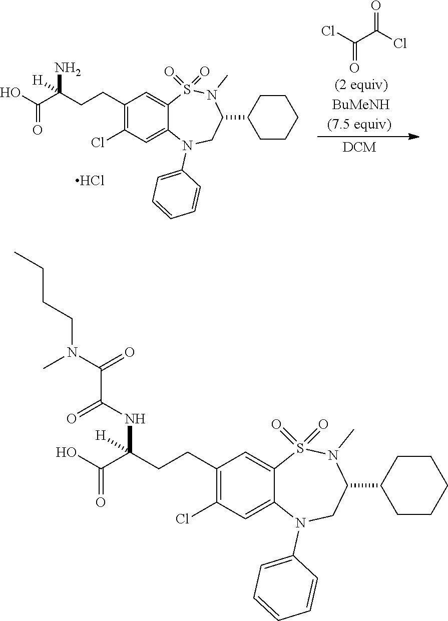

In a 1-dram vial, (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid hydrochloride (20.0 mg, 0.037 mmol, 1.0 equiv) was dissolved in DCM (0.2 mL, 0.18 M). In a second 1-dram vial equipped with a stir bar, oxalyl chloride (6.5 μL, 0.074 mmol, 2.0 equiv) was dissolved in DCM (0.3 mL). The azepane solution was then added dropwise to the oxalyl chloride solution at room temperature. The reaction was stirred for 4 h, monitored by LCMS. Upon complete conversion of the primary amine, N-methylbutan-1-amine (24 mg, 0.28 mmol, 7.5 equiv) was added dropwise. The reaction was stirred for an additional 30 min, monitored by LCMS. Upon sufficient conversion to the desired oxalamide, the reaction was quenched with 0.25 mL of formic acid, diluted with 1.0 mL of DMF, filtered through a syringe filter, and purified directly via RPHPLC to provide (S)-2-(2-(butyl(methyl)amino)-2-oxoacetamido)-4-((R)-7-chloro-3-cyclohexyl-2-methyl-1,1-dioxido-5-phenyl-2,3,4,5-tetrahydrobenzo[f][1,2,5]thiadiazepin-8-yl)butanoic acid. ESI MS m/z=647.5 [M+H]⁺.

The following compounds were prepared using a procedure analogous to that described for Ex. 32:

| Ex. # | Structure | MS |
|---|---|---|
| 33 | 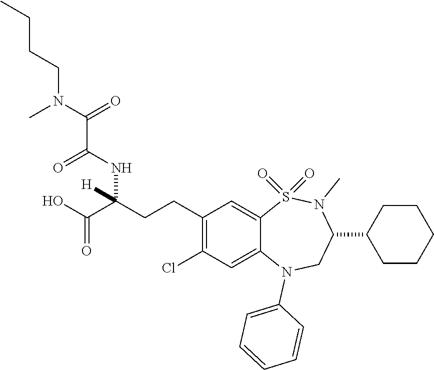 [SMILES: CCCCN(C)C(=O)C(=O)N[C@H](CCc1cc2c(cc1Cl)N(c1ccccc1)C[C@@H](C1CCCCC1)N(C)S2(=O)=O)C(=O)O] | [M + H]⁺ 647.4 |
| 34 | 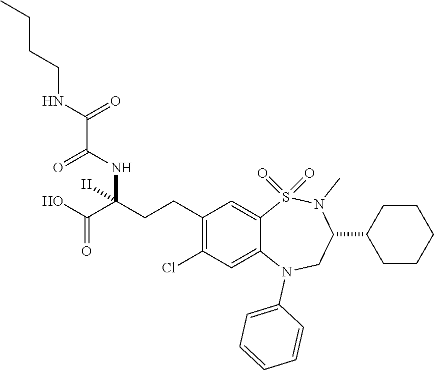 [SMILES: CCCCNC(=O)C(=O)N[C@@H](CCc1cc2c(cc1Cl)N(c1ccccc1)C[C@@H](C1CCCCC1)N(C)S2(=O)=O)C(=O)O] | [M + H]⁺ 633.4 |
| 35 | 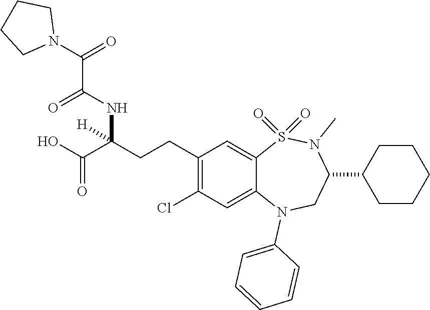 [SMILES: CN1[C@H](C2CCCCC2)CN(c2ccccc2)c2cc(Cl)c(CC[C@H](NC(=O)C(=O)N3CCCC3)C(=O)O)cc2S1(=O)=O] | [M + H]⁺ 631.4 |

### Ex. 36: Synthesis of (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-hexanamidobutanoic acid

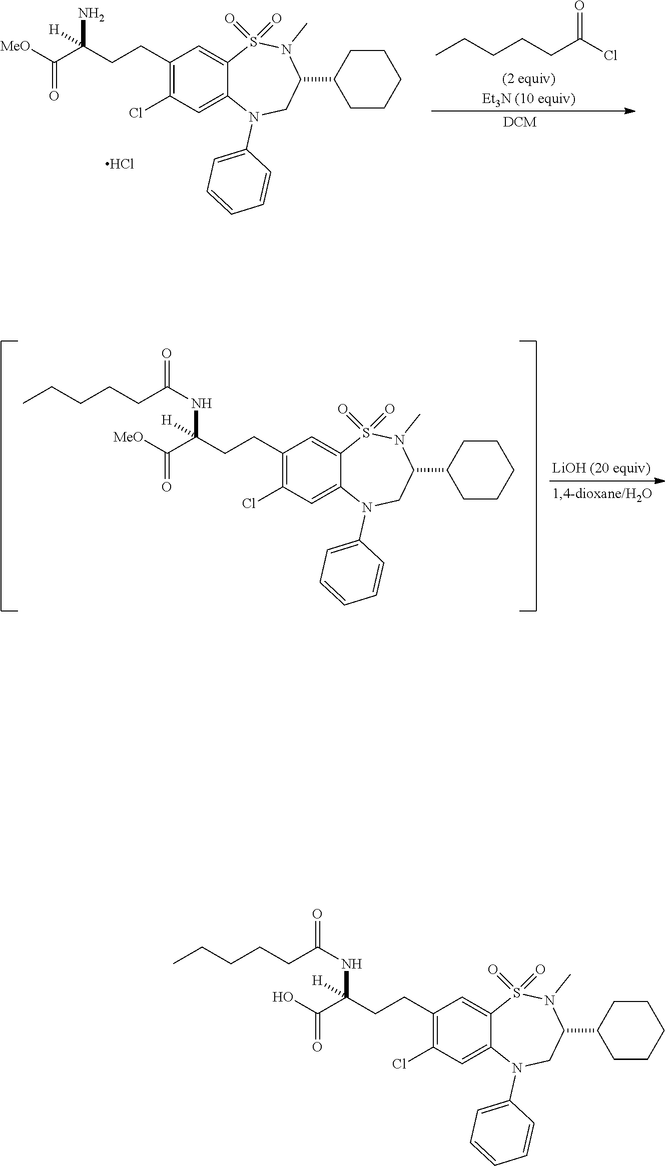

In a 1-dram vial equipped with a stir bar, methyl (S)-2-amino-4-((R)-7-chloro-3-cyclohexyl-2-methyl-1,1-dioxido-5-phenyl-2,3,4,5-tetrahydrobenzo[f][1,2,5]thiadiazepin-8-yl)butanoate hydrochloride (10.0 mg, 0.018 mmol, 1.0 equiv) was dissolved in DCM (0.50 mL, 0.03 M). To the reaction mixture, a solution of hexanoyl chloride (4.8 mg, 0.036 mmol, 2.0 equiv) in DCM (0.25 mL) was added dropwise. Then, Et₃N (25 μL, 0.18 mmol, 10 equiv) was added dropwise. The reaction was stirred for 15 min, monitored by LCMS. Upon complete conversion to the amide, the solvent was removed under a stream of nitrogen to provide crude methyl (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-hexanamidobutanoate (ESI MS m/z=618.4 [M+H]⁺). To the crude material, 1,4-dioxane (0.50 mL, 0.03 M), water (0.25 mL), and lithium hydroxide (8.6 mg, 0.36 mmol, 20 equiv) were added in that order. The reaction mixture was aggressively stirred at room temperature for 2 h, monitored by LCMS. Upon complete conversion to the acid, the reaction was quenched with 0.25 mL of formic acid, diluted with 1.0 mL of DMF, filtered through a syringe filter, and purified directly via RPHPLC to provide (S)-4-((R)-7-chloro-3-cyclohexyl-2-methyl-1,1-dioxido-5-phenyl-2,3,4,5-tetrahydrobenzo[f][1,2,5]thiadiazepin-8-yl)-2-hexanamidobutanoic acid. ESI MS m/z=604.4 [M+H]⁺.

The following compounds were prepared using a procedure analogous to that described for Ex. 36:

| Ex. # | Structure | MS |
|---|---|---|
| 37 | 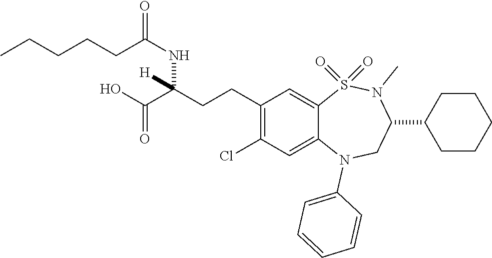 [SMILES: CCCCCC(=O)N[C@H](CCc1cc2c(cc1Cl)N(c1ccccc1)C[C@@H](C1CCCCC1)N(C)S2(=O)=O)C(=O)O] | [M + H]⁺ 604.5 |

### Ex. 38 Synthesis of (R)-3-((7-chloro-3-cyclohexyl-2-methyl-1,1-dioxido-5-phenyl-2,3,4,5-tetrahydrobenzo[f][1,2,5]thiadiazepin-8-yl)oxy)-2,2-dimethylpropanoic acid

### Step 1

In a 40 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (520 mg, 1.08 mmol, 1.0 equiv), bis(pinacolato)diboron (546.0 mg, 2.15 mmol, 2.0 equiv), potassium acetate (422.0 mg, 4.30 mmol, 4.0 equiv), and bis(triphenylphosphine)palladium(II) chloride (37.7 mg, 0.054 mmol, 0.05 equiv) were combined neat under a nitrogen atmosphere. Next, 1,4-dioxane (5.37 mL, 0.2M) was added, and the reaction mixture was heated at 80° C. for 12 h. Upon cooling to room temperature, analysis of the reaction mixture by LCMS indicated incomplete conversion to the desired arylboronic ester. Additional bis(pinacolato)diboron (273.0 mg, 1.08 mmol, 1.0 equiv) was added. The reaction mixture was re-heated to 80° C. for 16 h. Upon cooling to room temperature, the mixture was concentrated and purified by silica gel column chromatography (0 to 50% ethyl acetate/hexanes) to afford (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide as a white solid (350.5 mg, 0.66 mmol, 61% yield). ESI MS m/z=531.2 [M+H]⁺.

### Step 2

In a 40 mL vial equipped with a stir bar, (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (137 mg, 0.26 mmol, 1.0 equiv) was dissolved in THF (1.3 mL). The reaction mixture was cooled in an ice bath and 30 wt % aqueous hydrogen peroxide (5.0 equiv, 0.13 mL) was added, followed by water (0.5 mL) and sodium hydroxide (5.0 equiv, 0.22 mL, 6M). The reaction mixture was stirred for 1 h at room temperature. At this time, LCMS analysis indicated full conversion. The reaction mixture was diluted with water and quenched with HCL (1.2M, 0.30 mL). The aqueous phase was extracted with ethyl acetate. The combined organic layers were concentrated, and the crude residue was purified by RPHPLC to afford (R)-7-chloro-3-cyclohexyl-8-hydroxy-2-methyl-5-phenyl-2,3,4,5-tetrahydrobenzo[f][1,2,5]thiadiazepine 1,1-dioxide as a white solid (58.5 mg). ESI MS m/z=421.1 [M+H]⁺.

### Step 3

In a 40 mL vial equipped with a stir bar, (R)-7-chloro-3-cyclohexyl-8-hydroxy-2-methyl-5-phenyl-2,3,4,5-tetrahydrobenzo[f][1,2,5]thiadiazepine 1,1-dioxide (58.5 mg) was combined with methyl 3-hydroxy-2,2-dimethylpropanoate (22.0 mg, 0.17 mmol, 1.2 equiv) in THF (1.1 mL) under a nitrogen atmosphere. Next, triphenylphosphine (44.0 mg, 0.17 mmol, 1.2 equiv) was added, and the mixture was cooled in an ice and water bath. Diisopropyl azodicarboxylate (29.6 mg, 0.17 mmol) was added dropwise. After 2 h, water (0.25 mL) was added, followed by lithium hydroxide (33.3 mg, 1.39 mmol, 10.0 equiv). After stirring for 15 h, the reaction mixture was quenched by the addition of formic acid (0.5 mL) and concentrated. Purification of the residue by RPHPLC and lyophilization of the desired product from a mixture of acetonitrile and water afforded (R)-3-((7-chloro-3-cyclohexyl-2-methyl-1,1-dioxido-5-phenyl-2,3,4,5-tetrahydrobenzo[f][1,2,5]thiadiazepin-8-yl)oxy)-2,2-dimethylpropanoic acid (2.0 mg). ESI MS m/z=521.1 [M+H]⁺.

### Ex. 39 Synthesis of (S)-2-((R)-3-cyclohexyl-2-methyl-7-(methylamino)-1,1-dioxido-5-phenyl-2,3,4,5-tetrahydrobenzo[f][1,2,5]thiadiazepine-8-carboxamido)-2-(4-(trifluoromethyl)phenyl)acetic acid

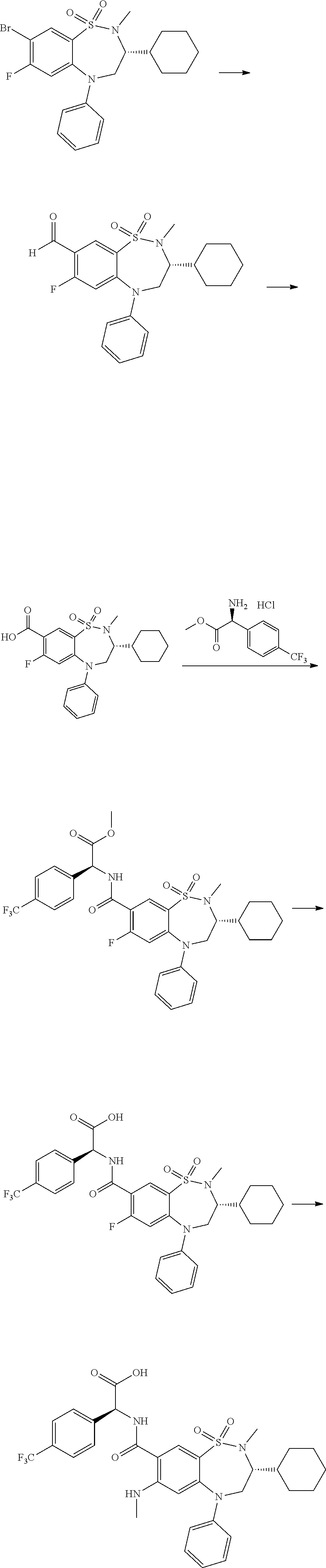

### Step 1

In a 40 ml vial equipped with stir bar, (R)-8-bromo-3-cyclohexyl-7-fluoro-2-methyl-5-phenyl-2,3,4,5-tetrahydrobenzo[f][1,2,5]thiadiazepine 1,1-dioxide (1.00 g, 2.14 mmol, 1.0 equiv) was dissolved in tetrahydrofuran (8.56 mL). The reaction mixture was cooled in a dry ice and acetone bath. Next, n-butyllithium (984 μL, 2.5M in hexanes, 1.15 equiv) was added dropwise. After stirring for 5 min at −78° C., N,N-dimethylformamide (4.97 mL, 30.0 equiv) was added. Reaction progress was monitored by LCMS analysis, and upon full conversion, the reaction mixture was quenched with 1.2M HCl. The aqueous phase was extracted with ethyl acetate, and the combined organic layers were dried over sodium sulfate. Upon concentration, the crude residue was purified by silica gel column chromatography (0 to 50% ethyl acetate/cyclohexane) to afford (R)-3-cyclohexyl-7-fluoro-2-methyl-5-phenyl-2,3,4,5-tetrahydrobenzo[f][1,2,5]thiadiazepine-8-carbaldehyde 1,1-dioxide (517.3 mg). ESI MS m/z=417.0 [M+H]⁺.

### Step 2

In a 250 mL round-bottomed flask equipped with a stir bar, (R)-3-cyclohexyl-7-fluoro-2-methyl-5-phenyl-2,3,4,5-tetrahydrobenzo[f][1,2,5]thiadiazepine-8-carbaldehyde 1,1-dioxide (517.3 mg, 1.24 mmol, 1.0 equiv) was dissolved in a solution of 2-methyl-2-butene in THF (2M, 12.42 mmol, 10.0 equiv). The mixture was then diluted with tert-butanol (12.4 mL) and a solution containing H₂NaPO₄ (447.0 mg, 3.73 mmol, 3.0 equiv) and sodium chlorite (280.8 mg, 2.48 mmol, 2.0 equiv) in water (6.0 mL) was added. The resulting mixture was stirred for 30 min at room temperature. At this time, LCMS analysis indicated high conversion to the desired carboxylic acid, and the mixture was diluted with 1.2M HCl. The aqueous phase was extracted with ethyl acetate, and the combined organic layers were dried over sodium sulfate. Upon concentration, the crude residue was purified by silica gel column chromatography to afford (R)-3-cyclohexyl-7-fluoro-2-methyl-5-phenyl-2,3,4,5-tetrahydrobenzo[f][1,2,5]thiadiazepine-8-carboxylic acid 1,1-dioxide (481.5 mg). ESI MS m/z=433.4 [M+H]⁺.

### Step 3

In a 4 mL vial equipped with a stir bar, (R)-3-cyclohexyl-7-fluoro-2-methyl-5-phenyl-2,3,4,5-tetrahydrobenzo[f][1,2,5]thiadiazepine-8-carboxylic acid 1,1-dioxide (50.0 mg, 0.12 mmol, 1.0 equiv) and methyl (S)-2-amino-2-(4-(trifluoromethyl)phenyl)acetate hydrochloride (34.0 mg, 0.13 mmol, 1.1 equiv) were dissolved in N,N-dimethylformamide (1.0 mL). Next, N,N-diisopropylethylamine (91 μL, 0.52 mmol, 4.5 equiv) was added, followed by HATU (48.0 mg, 0.13 mmol, 1.1 equiv). The resulting mixture was stirred at room temperature for 1 h 15 min before being filtered through a 0.45 micron syringe filter and purified directly by RPHPLC to afford methyl (S)-2-((R)-3-cyclohexyl-7-fluoro-2-methyl-1,1-dioxido-5-phenyl-2,3,4,5-tetrahydrobenzo[f][1,2,5]thiadiazepine-8-carboxamido)-2-(4-(trifluoromethyl)phenyl)acetate (48.1 mg). ESI MS m/z=648.3 [M+H]⁺.

### Step 4

In a 20 mL vial equipped with a stir bar, (S)-2-((R)-3-cyclohexyl-7-fluoro-2-methyl-1,1-dioxido-5-phenyl-2,3,4,5-tetrahydrobenzo[f][1,2,5]thiadiazepine-8-carboxamido)-2-(4-(trifluoromethyl)phenyl)acetate (48.1 mg) was dissolved in a mixture of 1,4-dioxane (1.0 mL) and water (0.5 mL). Lithium hydroxide (5.3 mg, 3.0 equiv) was added, and the mixture was stirred at room temperature for 1 h 20 min. The mixture was quenched with formic acid (300 μL), passed through a 0.45 micron syringe filter using N,N-dimethylformamide to rinse, and purified by RPHPLC to afford (S)-2-((R)-3-cyclohexyl-7-fluoro-2-methyl-1,1-dioxido-5-phenyl-2,3,4,5-tetrahydrobenzo[f][1,2,5]thiadiazepine-8-carboxamido)-2-(4-(trifluoromethyl)phenyl)acetic acid (21.5 mg). ESI MS m/z=634.4 [M+H]⁺.

### Step 5

In a 20 mL vial equipped with a stir bar, (S)-2-((R)-3-cyclohexyl-7-fluoro-2-methyl-1,1-dioxido-5-phenyl-2,3,4,5-tetrahydrobenzo[f][1,2,5]thiadiazepine-8-carboxamido)-2-(4-(trifluoromethyl)phenyl)acetic acid (19.2 mg) was dissolved in dimethyl sulfoxide (0.7 mL). Next a solution of methylamine in THF (2M, 0.15 mL, 10.0 equiv) was added, and the mixture was heated at 60° C. for 1 h. Upon cooling to room temperature the mixture was quenched with formic acid (300 μL) and passed through a 0.45 micron syringe filter before being purified by RPHPLC to afford (S)-2-((R)-3-cyclohexyl-2-methyl-7-(methylamino)-1,1-dioxido-5-phenyl-2,3,4,5-tetrahydrobenzo[f][1,2,5]thiadiazepine-8-carboxamido)-2-(4-(trifluoromethyl)phenyl)acetic acid (7.5 mg). ESI MS m/z=645.4 [M+H]⁺.

The following compounds were prepared using a procedure analogous to that described for Ex. 39:

| Ex. # | Structure | MS |
|---|---|---|
| 40 | 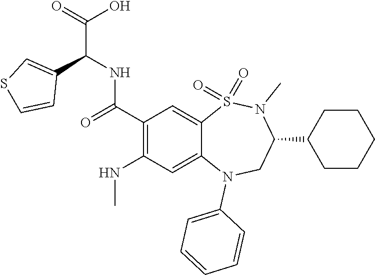 [SMILES: CNc1cc2c(cc1C(=O)N[C@H](C(=O)O)c1ccsc1)S(=O)(=O)N(C)[C@H](C1CCCCC1)CN2c1ccccc1] | [M + H]⁺ 583.0 |
| 41 | 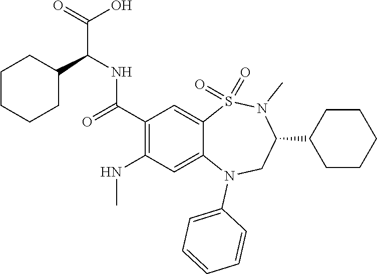 [SMILES: CNc1cc2c(cc1C(=O)N[C@H](C(=O)O)C1CCCCC1)S(=O)(=O)N(C)[C@H](C1CCCCC1)CN2c1ccccc1] | [M + H]⁺ 583.2 |
| 42 | 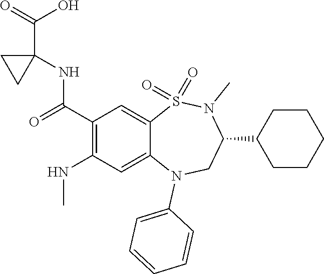 [SMILES: CNc1cc2c(cc1C(=O)NC1(C(=O)O)CC1)S(=O)(=O)N(C)[C@H](C1CCCCC1)CN2c1ccccc1] | [M + H]⁺ 527.3 |
| 43 | 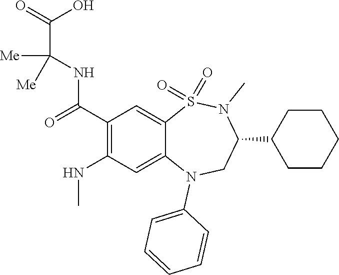 [SMILES: CNc1cc2c(cc1C(=O)NC(C)(C)C(=O)O)S(=O)(=O)N(C)[C@H](C1CCCCC1)CN2c1ccccc1] | [M + H]⁺ 529.4 |
| 44 | 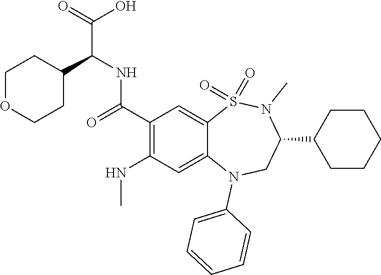 [SMILES: CNc1cc2c(cc1C(=O)N[C@H](C(=O)O)C1CCOCC1)S(=O)(=O)N(C)[C@H](C1CCCCC1)CN2c1ccccc1] | [M + H]⁺ 585.4 |
| 45 | 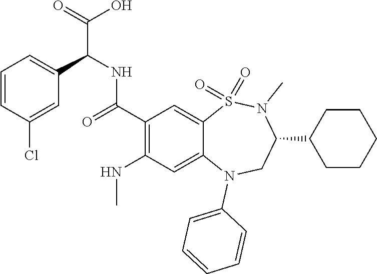 [SMILES: CNc1cc2c(cc1C(=O)N[C@H](C(=O)O)c1cccc(Cl)c1)S(=O)(=O)N(C)[C@H](C1CCCCC1)CN2c1ccccc1] | [M + H]⁺ 611.4 |
| 46 | 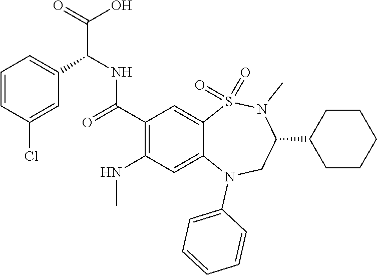 [SMILES: CNc1cc2c(cc1C(=O)N[C@@H](C(=O)O)c1cccc(Cl)c1)S(=O)(=O)N(C)[C@H](C1CCCCC1)CN2c1ccccc1] | [M + H]⁺ 611.4 |

### Biological Activity

### Methods:

### HBV Infection in HepG2-NTCP Cells

HepG2-NTCP A3 cells were maintained in DMEM media supplemented with GlutaMAX™, 10% fetal bovine serum, 1% penicillin/streptomycin, and 5 μg/mL puromycin at 37° C. in a humidified atmosphere with 5% CO₂ in a collagen-coated tissue culture flask.

HepG2-NTCP cells were seeded in 384 well plate containing 16,000 cells/well two days prior to the infection. On the day of infection, compounds were 3-fold serially diluted in DMSO and pre-incubated with HepG2-NTCP cells for two hours before purified HBV addition. HBV infection was carried out at 2000 GE/cell with 4% PEG, and the final concentration of DMSO is 0.5%. On day one post infection, HBV-containing media was aspirated, cells were washed once with PBS and then maintained in 2.5% DMSO containing media for the remainder of the assay.

Supernatants from infected HepG2-NTCP cells were collected at day 8 post infection, and the amount of viral antigen HBeAg was measured by HBeAg AlphaLISA detection kit (PerkinElmer) following the manufacturer's recommended protocol. Irrespective of readout, compound concentrations that reduce viral product accumulation in supernatants by 50% relative to DMSO controls (EC₅₀) are reported. EC₅₀ ranges are as follows: A<0.1 μM; B 0.1-1 μM; C>1 μM. A dash (-) indicates the compound was not tested.

TABLE 1
Summary of HBV Activities

| Example | Cellular | Example | Cellular |
|---|---|---|---|
| Number | EC₅₀ (μM) | Number | EC₅₀ (μM) |
| 1 | A | 2 | A |
| 3 | B | 4 | B |
| 5 | A | 6 | A |
| 7 | B | 8 | — |
| 9 | — | 10 | — |
| 11 | A | 12 | A |
| 13 | A | 14 | A |
| 15 | A | 16 | B |
| 17 | — | 18 | — |
| 19 | — | 20 | — |
| 21 | — | 22 | B |
| 23 | — | 24 | — |
| 25 | — | 26 | — |
| 27 | — | 28 | — |
| 29 | B | 30 | B |
| 31 | — | 32 | A |
| 33 | B | 34 | A |
| 35 | — | 36 | — |
| 37 | — | 38 | B |
| 39 | A | 40 | B |
| 41 | A | 42 | — |
| 43 | — | 44 | — |
| 45 | A | 46 | — |

Hepg2-NTCP preS1 Binding Competition Assay

Myristoylated preS1 peptide (2-48 aa) conjugated to a C-terminal FITC tag was synthesized to evaluate preS1 binding to NTCP-expressing cells. HepG2-NTCP cells seeded in 384-wells were pre-treated with compounds for 2 hours prior to the addition of FITC-labeled preS1 peptide. After co-incubation for 30 minutes, unbound FITC-preS1 peptide was washed twice with PBS, and the fluorescence of preS1-FITC bound to cell surface was detected by Envision plate reader. EC₅₀ ranges are as follows: A<0.1 μM; B 0.1-1 μM; C>1 μM. A dash (-) indicates the compound was not tested.

TABLE 4
Summary of HepG2-NTPC preS1 binding competition activities

| Example | Cellular | Example | Cellular |
|---|---|---|---|
| Number | EC₅₀ (μM) | Number | EC₅₀ (μM) |
| 1 | A | 2 | A |
| 3 | A | 4 | A |
| 5 | B | 6 | B |
| 7 | B | 8 | B |
| 9 | B | 10 | B |
| 11 | B | 12 | A |
| 13 | A | 14 | B |
| 15 | B | 16 | B |
| 17 | B | 18 | B |
| 19 | B | 20 | B |
| 21 | B | 22 | B |
| 23 | B | 24 | B |
| 25 | B | 26 | B |
| 27 | B | 28 | B |
| 29 | A | 30 | A |
| 31 | B | 32 | A |
| 33 | B | 34 | A |
| 35 | B | 36 | B |
| 37 | — | 38 | — |
| 39 | A | 40 | A |
| 41 | A | 42 | C |
| 43 | B | 44 | C |
| 45 | A | 46 | B |

While this invention has been particularly shown and described with references to preferred embodiments thereof, it will be understood by those skilled in the art that various changes in form and details may be made therein without departing from the scope of the invention encompassed by the appended claims.

1. A compound represented by Formula (I): 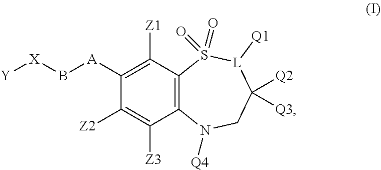
or a pharmaceutically acceptable salt thereof, wherein:
Q1, Q2, Q3, and Q4 are each independently selected from the group consisting of hydrogen, optionally substituted —C₁-C₆ alkyl, optionally substituted —C₂-C₆ alkenyl, optionally substituted —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, and optionally substituted heteroaryl;
alternatively, Q1 and Q2, or Q1 and Q3 are taken together with the atoms to which they are attached to form an optionally substituted 3-8 membered heterocyclic or carbocyclic ring containing 0, 1, 2, or 3 double bonds;
alternatively, Q2 and Q3 are taken together with the carbon atom to which they are attached to form an optionally substituted 3-8 membered heterocyclic or carbocyclic ring containing 0, 1, 2, or 3 double bonds;
L is CR₁₄ or N;
R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy;
Z1, Z2, and Z3 are each independently selected from the group consisting of:
1) hydrogen;
2) halogen;
3) —OH;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₈ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁)S(O)₂(R₁₂);
21) —N(R₁)(R₁₂);
22) —N(R₁₃)C(O)N(R₁)(R₁₂);
23) —N(R₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
wherein R₁₁, R₁₂, and R₁₃, are each independently selected from the group consisting of hydrogen, optionally substituted —C₁-C₁₂ alkyl, optionally substituted —C₂-C₁₂ alkenyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; alternatively, R₁₁ and R₁₂ are taken together with the nitrogen atom to which they attached to form an optionally substituted 3-8 membered heterocyclic containing 0, 1, 2, or 3 double bonds;
A is absent or selected from the group consisting of O, NR₁₅, CR₁₆R₁₆′, S(O)₂, C(O), optionally substituted —C₃-C₈ cycloalkyl, optionally substituted —C₃-C₈ cycloalkenyl, and optionally substituted 3- to 8-membered heterocycloalkyl;
B is absent or selected from the group consisting of O, NR₁₅, CR₁₇R₁₇′, optionally substituted —C₃-C₈ cycloalkyl, optionally substituted —C₃-C₈ cycloalkenyl, and optionally substituted 3- to 8-membered heterocycloalkyl;
X is absent or selected from the group consisting of C(O), O, NR₁₅, SO₂, CR₁₈R₁₈′, and optionally substituted —C₃-C₈ cycloalkyl, optionally substituted —C₃-C₈ cycloalkenyl, and optionally substituted 3- to 8-membered heterocycloalkyl;
alternatively, when A is CR₁₆R₁₆′, R₁₆ and R₁₆′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
alternatively, when B is CR₁₇R₁₇′, R₁₇ and R₁₇′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
alternatively, when X is CR₁₈R₁₈′, R₁₈ and R₁₈′ are taken together with the carbon atoms to which they are attached to form an optionally substituted —C₃-C₈ cycloalkyl, optionally substituted —C₃-C₈ cycloalkenyl, or optionally substituted 3- to 12-membered heterocycloalkyl;
alternatively, A and B are taken together to form 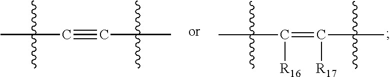
R₁₅, R₁₆, R₁₆′, R₁₇, R₁₇′, R₁₈ and R₁₈′ are each independently selected from the group consisting of:
1) hydrogen;
2) halogen;
3) hydroxy;
4) cyano;
5) —N(R₁₁)(R₁₂);
6) —N(R₁₃)C(O)N(R₁₁)(R₁₂);
7) —N(R₁₃)C(O)C(O)N(R₁₁)(R₁₂);
8) —N(R₁)C(O)(R₁₂);
9) —N(R₁₁)C(O)₂(R₁₂);
10) —N(R₁₃)S(O)₂N(R₁₁)(R₁₂);
11) —N(R₁)S(O)₂(R₁₂);
12) Optionally substituted —C₁-C₈ alkyl;
13) Optionally substituted —C₃-C₈ cycloalkyl;
14) Optionally substituted —C₃-C₈ cycloalkenyl,
15) Optionally substituted 3- to 12-membered heterocycloalkyl;
16) Optionally substituted aryl;
17) Optionally substituted heteroaryl; and
18) Optionally substituted alkoxy;
Y is —CO₂H, —PO₃H₂, —SO₃H, —B(OH)₂, —C(O)NHR₂₁, —SO₂NHR₂₁, —SO₂NHC(O)R₂₁, —C(O)NHSO₂R₂₁, tetrazolyl, or triazolyl; and
R₂₁ is independently selected from the group consisting of hydrogen, optionally substituted —C₁-C₈ alkyl, optionally substituted C₂-C₈ alkenyl, optionally substituted C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl.

2. The compound of claim 1, represented by Formula (V), or a pharmaceutically acceptable salt thereof: 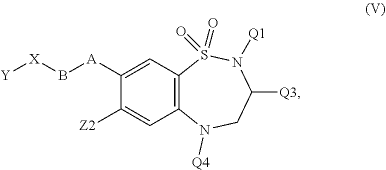
wherein Q1, Q3, Q4, Z2, A, B, X, and Y are as defined in claim 1.

3. The compound of claim 1, represented by one of Formulae (IX-1)˜(IX-4), or a pharmaceutically acceptable salt thereof: 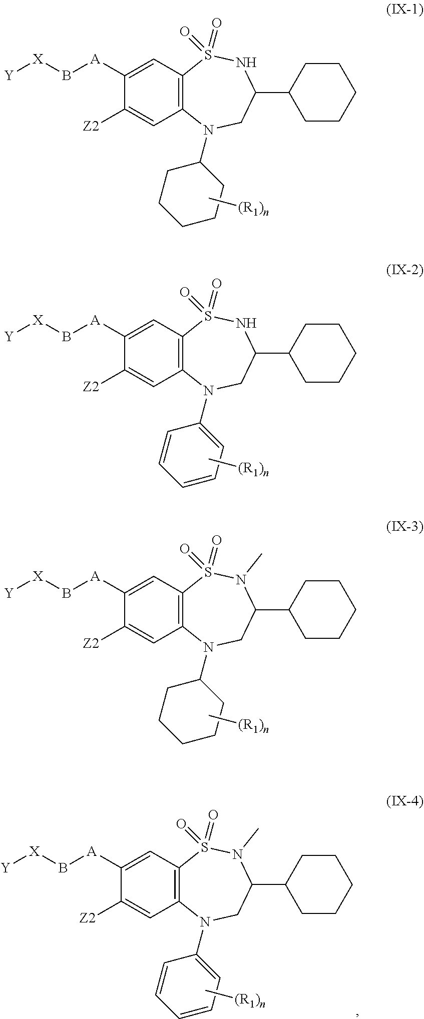
wherein each R₁ is independently selected from the group consisting of:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
n is 0, 1, 2 or 3; and Z2, A, B, X, and Y are as defined in claim 1.

4. The compound of claim 1, represented by one of Formulae (XIII-1)˜(XIII-10), or a pharmaceutically acceptable salt thereof: 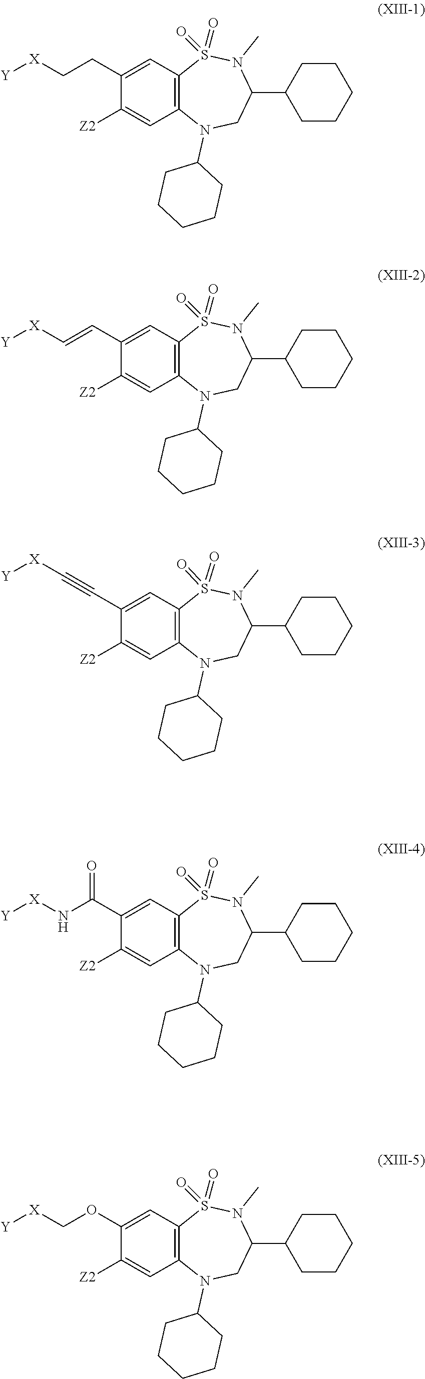 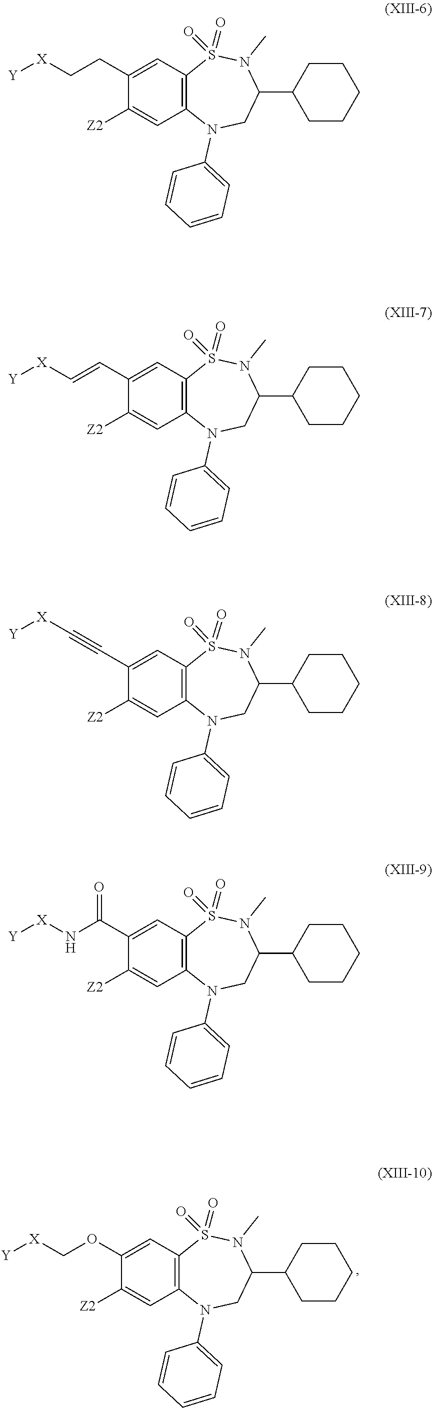
wherein Z2, X, and Y are as defined in claim 1.

5. The compound of claim 1, selected from the compounds set forth below, or a pharmaceutically acceptable salt thereof:

| Compound | Structure |
|---|---|
| 1 | 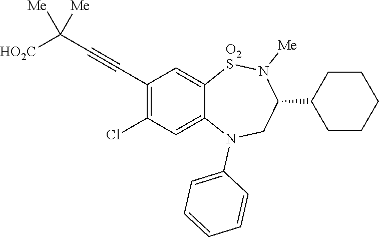 [SMILES: CN1[C@H](C2CCCCC2)CN(c2ccccc2)c2cc(Cl)c(C#CC(C)(C)C(=O)O)cc2S1(=O)=O] |
| 2 | 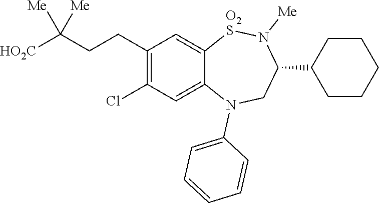 [SMILES: CN1[C@H](C2CCCCC2)CN(c2ccccc2)c2cc(Cl)c(CCC(C)(C)C(=O)O)cc2S1(=O)=O] |
| 3 | 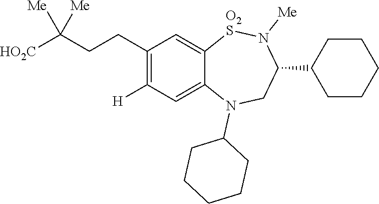 [SMILES: CN1[C@H](C2CCCCC2)CN(C2CCCCC2)c2ccc(CCC(C)(C)C(=O)O)cc2S1(=O)=O] |
| 4 | 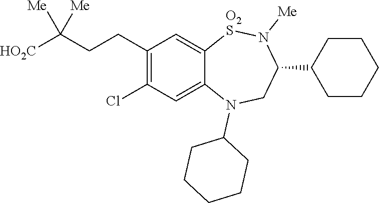 [SMILES: CN1[C@H](C2CCCCC2)CN(C2CCCCC2)c2cc(Cl)c(CCC(C)(C)C(=O)O)cc2S1(=O)=O] |
| 5 | 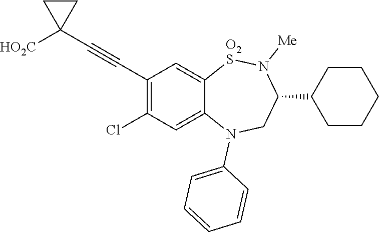 [SMILES: CN1[C@H](C2CCCCC2)CN(c2ccccc2)c2cc(Cl)c(C#CC3(C(=O)O)CC3)cc2S1(=O)=O] |
| 6 | 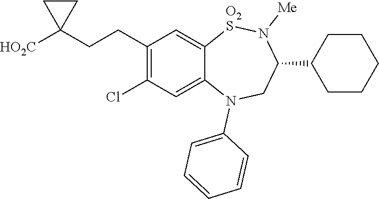 [SMILES: CN1[C@H](C2CCCCC2)CN(c2ccccc2)c2cc(Cl)c(CCC3(C(=O)O)CC3)cc2S1(=O)=O] |
| 7 | 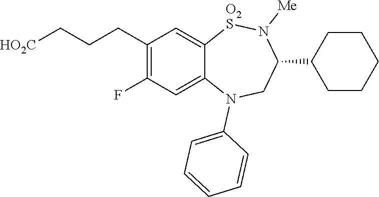 [SMILES: CN1[C@H](C2CCCCC2)CN(c2ccccc2)c2cc(F)c(CCCC(=O)O)cc2S1(=O)=O] |
| 8 | 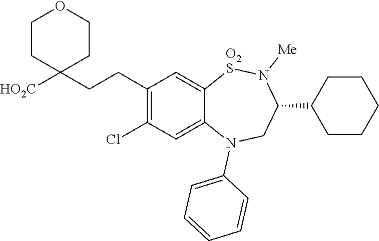 [SMILES: CN1[C@H](C2CCCCC2)CN(c2ccccc2)c2cc(Cl)c(CCC3(C(=O)O)CCOCC3)cc2S1(=O)=O] |
| 9 | 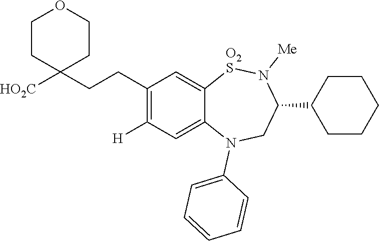 [SMILES: CN1[C@H](C2CCCCC2)CN(c2ccccc2)c2ccc(CCC3(C(=O)O)CCOCC3)cc2S1(=O)=O] |
| 10 | 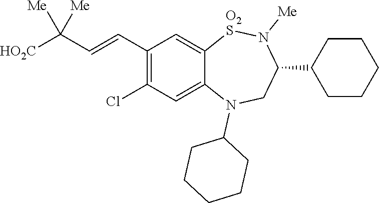 [SMILES: CN1[C@H](C2CCCCC2)CN(C2CCCCC2)c2cc(Cl)c(/C=C/C(C)(C)C(=O)O)cc2S1(=O)=O] |
| 11 | 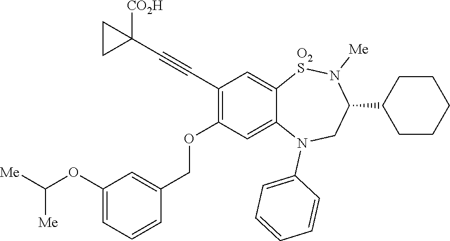 [SMILES: CC(C)Oc1cccc(COc2cc3c(cc2C#CC2(C(=O)O)CC2)S(=O)(=O)N(C)[C@H](C2CCCCC2)CN3c2ccccc2)c1] |
| 12 | 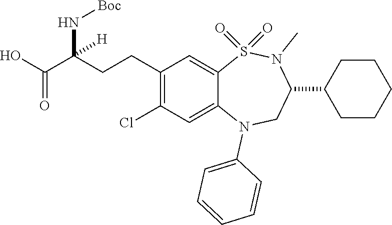 [SMILES: CN1[C@H](C2CCCCC2)CN(c2ccccc2)c2cc(Cl)c(CC[C@H](NC(=O)OC(C)(C)C)C(=O)O)cc2S1(=O)=O] |
| 13 | 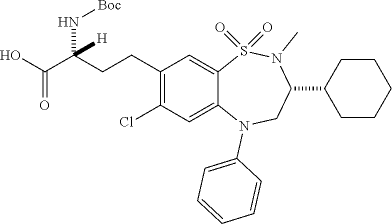 [SMILES: CN1[C@H](C2CCCCC2)CN(c2ccccc2)c2cc(Cl)c(CC[C@@H](NC(=O)OC(C)(C)C)C(=O)O)cc2S1(=O)=O] |
| 14 | 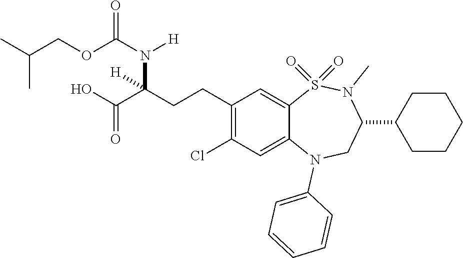 [SMILES: CC(C)COC(=O)N[C@@H](CCc1cc2c(cc1Cl)N(c1ccccc1)C[C@@H](C1CCCCC1)N(C)S2(=O)=O)C(=O)O] |
| 15 | 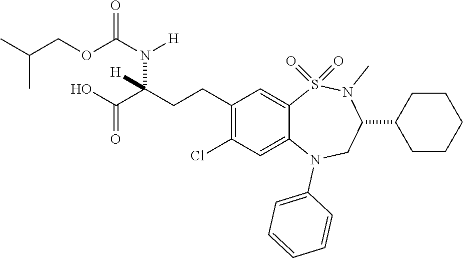 [SMILES: CC(C)COC(=O)N[C@H](CCc1cc2c(cc1Cl)N(c1ccccc1)C[C@@H](C1CCCCC1)N(C)S2(=O)=O)C(=O)O] |
| 16 | 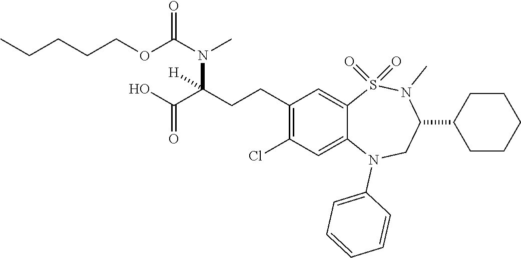 [SMILES: CCCCCOC(=O)N(C)[C@@H](CCc1cc2c(cc1Cl)N(c1ccccc1)C[C@@H](C1CCCCC1)N(C)S2(=O)=O)C(=O)O] |
| 17 | 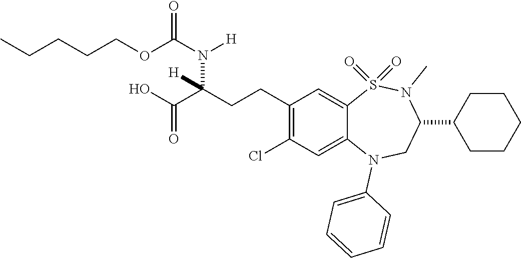 [SMILES: CCCCCOC(=O)N[C@H](CCc1cc2c(cc1Cl)N(c1ccccc1)C[C@@H](C1CCCCC1)N(C)S2(=O)=O)C(=O)O] |
| 18 | 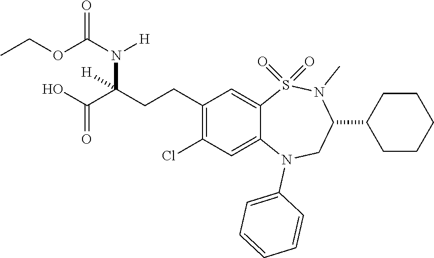 [SMILES: CCOC(=O)N[C@@H](CCc1cc2c(cc1Cl)N(c1ccccc1)C[C@@H](C1CCCCC1)N(C)S2(=O)=O)C(=O)O] |
| 19 | 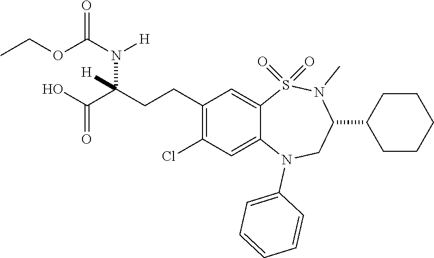 [SMILES: CCOC(=O)N[C@H](CCc1cc2c(cc1Cl)N(c1ccccc1)C[C@@H](C1CCCCC1)N(C)S2(=O)=O)C(=O)O] |
| 20 | 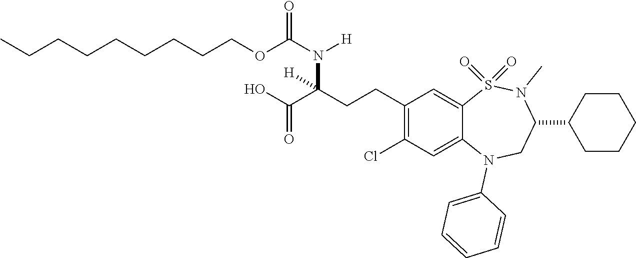 [SMILES: CCCCCCCCCOC(=O)N[C@@H](CCc1cc2c(cc1Cl)N(c1ccccc1)C[C@@H](C1CCCCC1)N(C)S2(=O)=O)C(=O)O] |
| 21 | 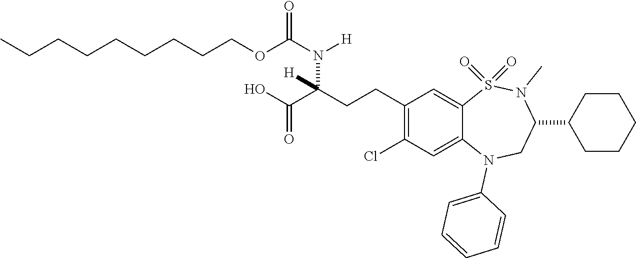 [SMILES: CCCCCCCCCOC(=O)N[C@H](CCc1cc2c(cc1Cl)N(c1ccccc1)C[C@@H](C1CCCCC1)N(C)S2(=O)=O)C(=O)O] |
| 22 | 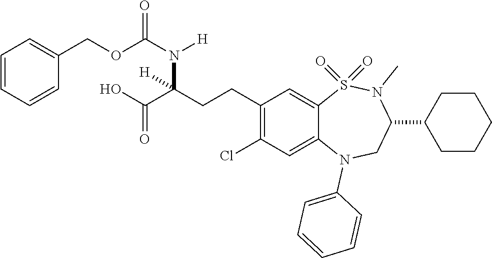 [SMILES: CN1[C@H](C2CCCCC2)CN(c2ccccc2)c2cc(Cl)c(CC[C@H](NC(=O)OCc3ccccc3)C(=O)O)cc2S1(=O)=O] |
| 23 | 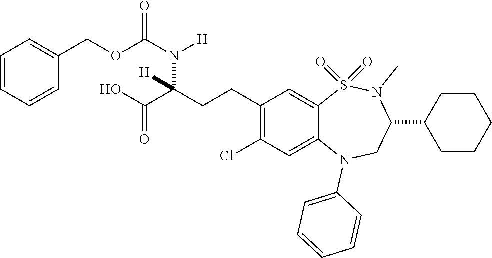 [SMILES: CN1[C@H](C2CCCCC2)CN(c2ccccc2)c2cc(Cl)c(CC[C@@H](NC(=O)OCc3ccccc3)C(=O)O)cc2S1(=O)=O] |
| 24 | 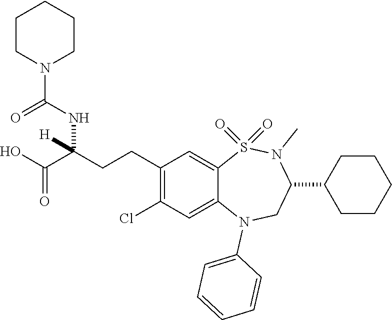 [SMILES: CN1[C@H](C2CCCCC2)CN(c2ccccc2)c2cc(Cl)c(CC[C@@H](NC(=O)N3CCCCC3)C(=O)O)cc2S1(=O)=O] |
| 25 | 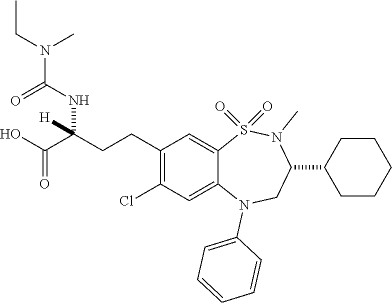 [SMILES: CCN(C)C(=O)N[C@H](CCc1cc2c(cc1Cl)N(c1ccccc1)C[C@@H](C1CCCCC1)N(C)S2(=O)=O)C(=O)O] |
| 26 | 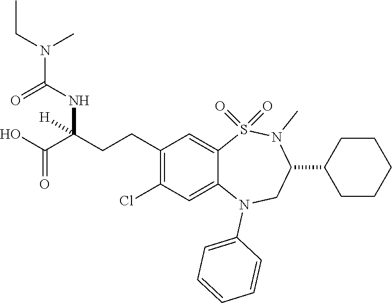 [SMILES: CCN(C)C(=O)N[C@@H](CCc1cc2c(cc1Cl)N(c1ccccc1)C[C@@H](C1CCCCC1)N(C)S2(=O)=O)C(=O)O] |
| 27 | 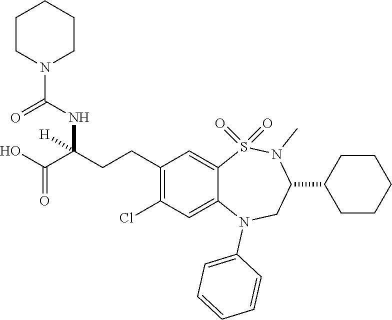 [SMILES: CN1[C@H](C2CCCCC2)CN(c2ccccc2)c2cc(Cl)c(CC[C@H](NC(=O)N3CCCCC3)C(=O)O)cc2S1(=O)=O] |
| 28 | 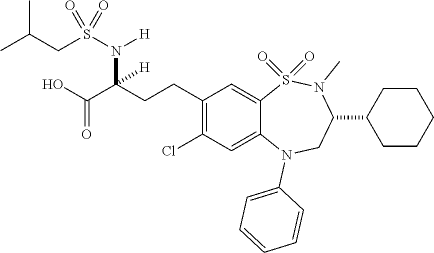 [SMILES: CC(C)CS(=O)(=O)N[C@@H](CCc1cc2c(cc1Cl)N(c1ccccc1)C[C@@H](C1CCCCC1)N(C)S2(=O)=O)C(=O)O] |
| 29 | 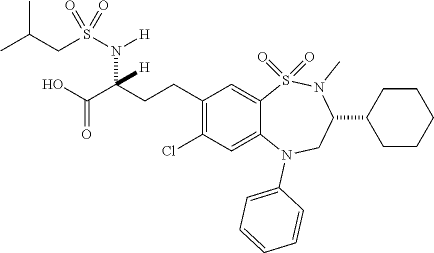 [SMILES: CC(C)CS(=O)(=O)N[C@H](CCc1cc2c(cc1Cl)N(c1ccccc1)C[C@@H](C1CCCCC1)N(C)S2(=O)=O)C(=O)O] |
| 30 | 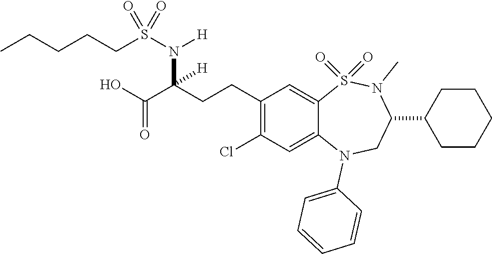 [SMILES: CCCCCS(=O)(=O)N[C@@H](CCc1cc2c(cc1Cl)N(c1ccccc1)C[C@@H](C1CCCCC1)N(C)S2(=O)=O)C(=O)O] |
| 31 | 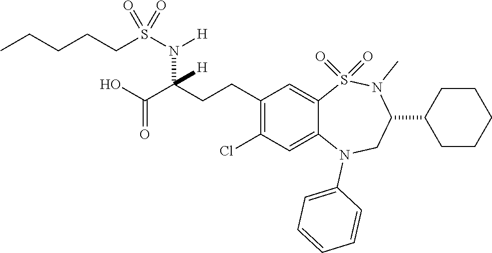 [SMILES: CCCCCS(=O)(=O)N[C@H](CCc1cc2c(cc1Cl)N(c1ccccc1)C[C@@H](C1CCCCC1)N(C)S2(=O)=O)C(=O)O] |
| 32 | 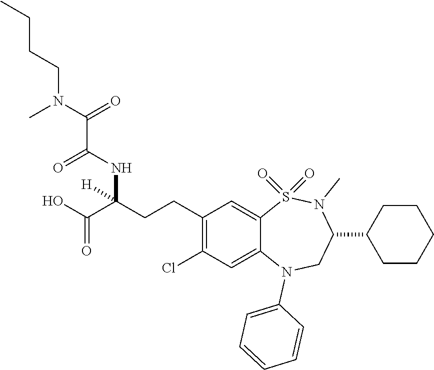 [SMILES: CCCCN(C)C(=O)C(=O)N[C@@H](CCc1cc2c(cc1Cl)N(c1ccccc1)C[C@@H](C1CCCCC1)N(C)S2(=O)=O)C(=O)O] |
| 33 | 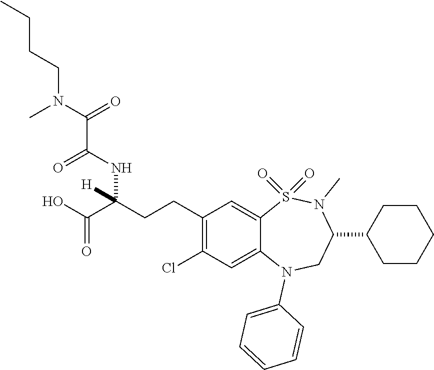 [SMILES: CCCCN(C)C(=O)C(=O)N[C@H](CCc1cc2c(cc1Cl)N(c1ccccc1)C[C@@H](C1CCCCC1)N(C)S2(=O)=O)C(=O)O] |
| 34 | 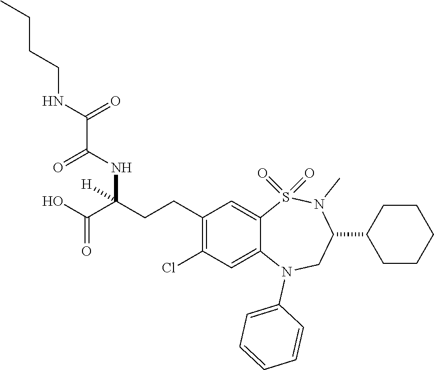 [SMILES: CCCCNC(=O)C(=O)N[C@@H](CCc1cc2c(cc1Cl)N(c1ccccc1)C[C@@H](C1CCCCC1)N(C)S2(=O)=O)C(=O)O] |
| 35 | 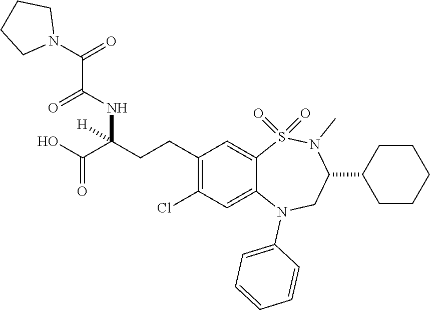 [SMILES: CN1[C@H](C2CCCCC2)CN(c2ccccc2)c2cc(Cl)c(CC[C@H](NC(=O)C(=O)N3CCCC3)C(=O)O)cc2S1(=O)=O] |
| 36 | 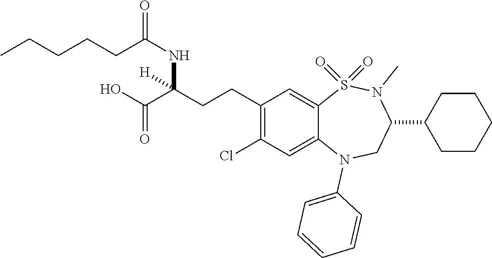 [SMILES: CCCCCC(=O)N[C@@H](CCc1cc2c(cc1Cl)N(c1ccccc1)C[C@@H](C1CCCCC1)N(C)S2(=O)=O)C(=O)O] |
| 37 | 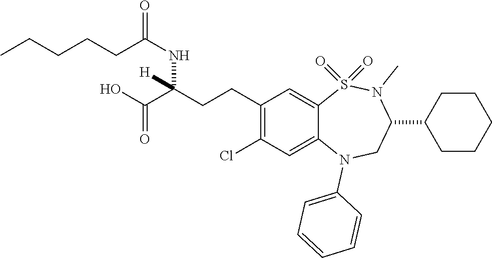 [SMILES: CCCCCC(=O)N[C@H](CCc1cc2c(cc1Cl)N(c1ccccc1)C[C@@H](C1CCCCC1)N(C)S2(=O)=O)C(=O)O] |
| 38 | 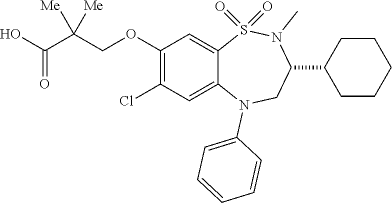 [SMILES: CN1[C@H](C2CCCCC2)CN(c2ccccc2)c2cc(Cl)c(OCC(C)(C)C(=O)O)cc2S1(=O)=O] |
| 39 | 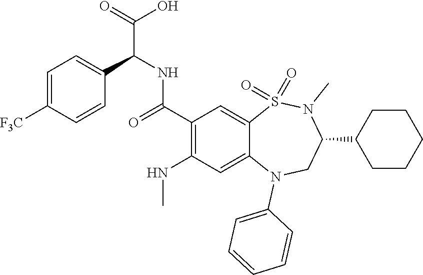 [SMILES: CNc1cc2c(cc1C(=O)N[C@H](C(=O)O)c1ccc(C(F)(F)F)cc1)S(=O)(=O)N(C)[C@H](C1CCCCC1)CN2c1ccccc1] |
| 40 | 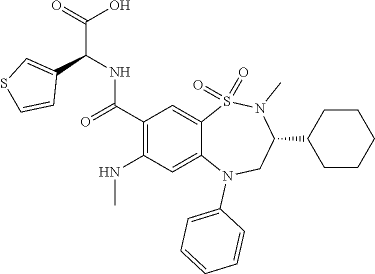 [SMILES: CNc1cc2c(cc1C(=O)N[C@H](C(=O)O)c1ccsc1)S(=O)(=O)N(C)[C@H](C1CCCCC1)CN2c1ccccc1] |
| 41 | 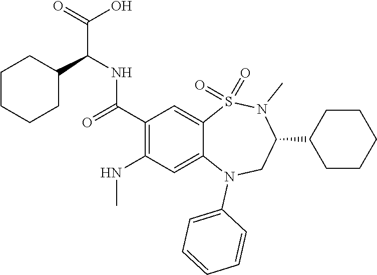 [SMILES: CNc1cc2c(cc1C(=O)N[C@H](C(=O)O)C1CCCCC1)S(=O)(=O)N(C)[C@H](C1CCCCC1)CN2c1ccccc1] |
| 42 | 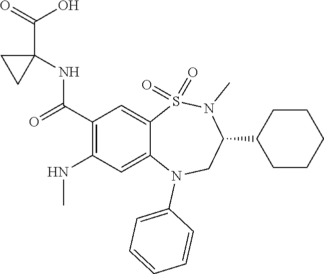 [SMILES: CNc1cc2c(cc1C(=O)NC1(C(=O)O)CC1)S(=O)(=O)N(C)[C@H](C1CCCCC1)CN2c1ccccc1] |
| 43 | 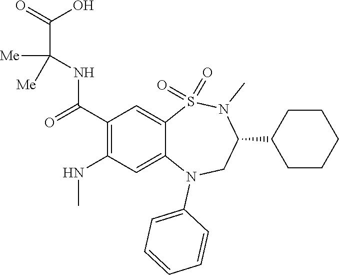 [SMILES: CNc1cc2c(cc1C(=O)NC(C)(C)C(=O)O)S(=O)(=O)N(C)[C@H](C1CCCCC1)CN2c1ccccc1] |
| 44 | 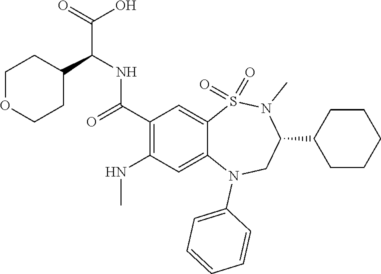 [SMILES: CNc1cc2c(cc1C(=O)N[C@H](C(=O)O)C1CCOCC1)S(=O)(=O)N(C)[C@H](C1CCCCC1)CN2c1ccccc1] |
| 45 | 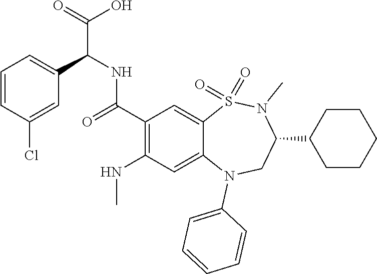 [SMILES: CNc1cc2c(cc1C(=O)N[C@H](C(=O)O)c1cccc(Cl)c1)S(=O)(=O)N(C)[C@H](C1CCCCC1)CN2c1ccccc1] |
| 46 | 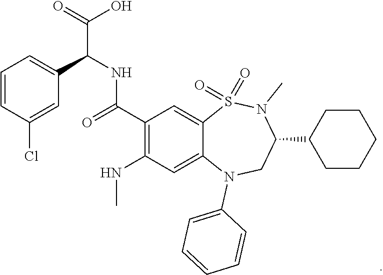 [SMILES: CNc1cc2c(cc1C(=O)N[C@H](C(=O)O)c1cccc(Cl)c1)S(=O)(=O)N(C)[C@H](C1CCCCC1)CN2c1ccccc1] |

6. A pharmaceutical composition, comprising a compound according to claim 1, and a pharmaceutically acceptable carrier or excipient.

7. A method of treating or preventing an HBV and/or HDV infection in a subject in need thereof, comprising administering to the subject a therapeutically effective amount of a compound or a combination of compounds according to claim 1.

8. The method of claim 7, further comprising administering to the subject an additional therapeutic agent selected from the group consisting of a HBV polymerase inhibitor, interferon, viral entry inhibitor, viral maturation inhibitor, literature-described capsid assembly modulator, reverse transcriptase inhibitor, TLR-agonist, inducer of cellular viral RNA sensor, therapeutic vaccine, and agents of distinct or unknown mechanism, and a combination thereof.

9. The method of claim 8, wherein the compound and the additional therapeutic agent are co-formulated.

10. The method of claim 8, wherein the compound and the additional therapeutic agent are co-administered.

11. The method of claim 8, wherein the additional therapeutic agent is administered at a lower dose or frequency compared to the dose or frequency of the additional therapeutic agent that is required to treat an HBV and/or HDV infection when administered alone.

12. The method of claim 8, wherein the subject is refractory to at least one compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

13. The method of claim 8, wherein the method reduces viral load in the subject to a greater extent compared to the administering of a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

14. The method of claim 8, wherein the method results in a lower incidence of viral mutation and/or viral resistance than the treatment with a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.
